(19) United States
(12) Patent Application Publication
(10) Pub. No.: US 2016/0178644 A1
(43) Pub. Date: Jun. 23, 2016

(54) METHODS FOR DIAGNOSING AND TREATING INFLAMMATORY BOWEL DISEASE

(71) Applicant: GENENTECH, INC., South San Francisco, CA (US)

(72) Inventors: Jason A. Hackney, San Carlos, CA (US); Mary Keir, San Francisco, CA (US); Lilyan Yi Tian Wright, Foster City, CA (US)

(73) Assignee: GENENTECH, INC., South San Francisco, CA (US)

(21) Appl. No.: 14/944,147

(22) Filed: Nov. 17, 2015

Related U.S. Application Data
(63) Continuation of application No. PCT/US2014/038434, filed on May 16, 2014.
(60) Provisional application No. 61/824,661, filed on May 17, 2013.

(51) Int. Cl. G01N 33/68; C12Q 1/68; C07K 16/24

(52) U.S. Cl. CPC G01N 33/6893; C07K 16/244; C12Q 1/6883; C12Q 2600/158; G01N 2800/065

(57) ABSTRACT

Biomarkers for diagnosing inflammatory bowel disease, including Crohn's disease, including fibrotic or fibrostenotic Crohn's disease, and ulcerative colitis and methods of using such biomarkers are provided. In addition, methods of treating gastrointestinal inflammatory disorders such as inflammatory bowel diseases including ulcerative colitis and Crohn's disease, including fibrotic or fibrostenotic Crohn's disease, are provided.

### CROSS REFERENCE TO RELATED APPLICATION

This application is a continuation application of PCT/US2014/038434, filed on May 16, 2014, which claims the benefit of priority of provisional U.S. Application No. 61/824,661 filed May 17, 2013 which is hereby incorporated by reference in its entirety.

### FIELD

Biomarkers for diagnosing inflammatory bowel disease, including Crohn's disease, including fibrotic or fibrostenotic Crohn's disease, and ulcerative colitis and methods of using such biomarkers are provided. In addition, methods of treating gastrointestinal inflammatory disorders such as inflammatory bowel diseases including ulcerative colitis and Crohn's disease, including fibrotic or fibrostenotic Crohn's disease, are provided.

### BACKGROUND

Inflammatory bowel disease (IBD) is a chronic inflammatory autoimmune condition of the gastrointestinal (GI) tract, which presents clinically as either ulcerative colitis (UC) or Crohn's disease (CD). CD is a chronic transmural inflammatory disease with the potential to affect any part of the entire GI tract, and UC is a mucosal inflammation of the colon. Both conditions are characterized clinically by frequent bowel motions, malnutrition, and dehydration, with disruption in the activities of daily living. Chronic inflammation may lead to fibrosis in a subset of CD patients, with complications including strictures and fistulae and may require repeated surgery. UC, less frequently, may be complicated by severe bloody diarrhea and toxic megacolon, also requiring surgery. Both IBD conditions are associated with an increased risk for malignancy of the GI tract. The etiology of IBD is complex, and many aspects of the pathogenesis remain unclear.

Activation of intestinal myofibroblasts may play a key role in intestinal fibrosis through increased deposition of collagen and extracellular matrix proteins. The role of inflammatory cytokines in this process is not understood.

The interleukin-1 (IL-1) and IL-18 family of cytokines are related by mechanism of origin, receptor structure, and signal transduction pathways utilized. These cytokines are synthesized as precursor molecules and cleaved by the enzyme caspase-1 before or during release from the cell. The NALP-3 inflammasome is of crucial importance in generating active caspase-1 (Cassel et al., 2009; Ferrero-Miliani et al., 2007). The IL-1 family contains two agonists, IL-1α and IL-1β, a specific inhibitor, IL-1 receptor antagonist (IL-1Ra), and two receptors, the biologically active type IL-1R and inactive type II IL-1R (Arend et al., 2008). Both IL-1RI and IL-33R utilize the same interacting accessory protein (IL-1RAcP). The balance between IL-1 and IL-1Ra is important in preventing disease in various organs, and excess production of IL-1 has been implicated in many human diseases. The IL-18 family also contains a specific inhibitor, the IL-18-binding protein (IL-18BP), which binds IL-18 in the fluid phase. The IL-18 receptor is similar to the IL-1 receptor complex, including a single ligand-binding chain and a different interacting accessory protein. IL-18 provides an important link between the innate and adaptive immune responses.

Inflammasome activation and IL-1β/IL-18 processing and secretion may be involved in disease progression. Genome-wide association studies indicate a role for the inflammasome in inflammatory bowel disease (IBD). Patients with polymorphisms in the inflammasome-compound NALP-3 are reportedly at increased risk for Crohn's disease (Ferrero-Miliani et al., 2007; Villani et al., 2009). In addition, polymorphisms in autophagy components Atg16l1 and IRGM that control caspase-1 activation and IL-1β/IL-18 processing have been reportedly linked to Crohn's disease (Baldassano et al., 2007; Cadwell et al., 2008; Kuballa et al., 2008; Saitoh et al., 2008). Independent studies have reported increased serum levels of IL-1β and IL-18 in patients with IBD (Ludwiczek et al., 2005; Ludwiczek et al., 2004; Monteleone et al., 1999). Studies in humans have been further supported by preclinical studies. Blockade of IL-18 or IL-1β reportedly leads to amelioration of clinical scores in preclinical models of the disease (Ten Hove et al., 2001).

The treatment of moderate to severe IBD poses significant challenges to treating physicians, because conventional therapy with corticosteroids and immunomodulator therapy (e.g., azathioprine, 6 mercaptopurine, and methotrexate) is associated with side effects and intolerance and has not shown proven benefit in maintenance therapy (steroids). Monoclonal antibodies targeting tumor necrosis factor alpha (TNF-α), such as infliximab (a chimeric antibody) and adalimumab (a fully human antibody), are currently used in the management of CD. Infliximab has also shown efficacy and has been approved for use in UC. However, approximately 10%-20% of patients with CD are primary nonresponders to anti TNF therapy, and another ˜20%-30% of CD patients lose response over time (Schnitzler et al., Gut 58:492-500 (2009)). Other adverse events (AEs) associated with anti TNFs include elevated rates of bacterial infection, including tuberculosis, and, more rarely, lymphoma and demyelination (Chang et al., Nat Clin Pract Gastroenterol Hepatology 3:220 (2006); Hoentjen et al., World J. Gastroenterol. 15(17):2067 (2009)). No currently available therapy achieves sustained remission in more than 20%-30% of IBD patients with chronic disease (Hanauer et al., Lancet 359:1541-49 (2002); Sandborn et al., N Engl J Med 353:1912-25 (2005)). In addition, most patients do not achieve sustained steroid-free remission and mucosal healing, clinical outcomes that correlate with true disease modification. Therefore, there is a need to develop more targeted therapy in IBD that is optimized for chronic use: an improved safety profile with sustained remission, particularly steroid-free remission and prevention of long-term complications in a greater proportion of patients, including those patients who either never respond to an anti TNF therapeutic agent or lose response over time.

It is often unknown, prior to treatment, whether a patient will respond to a particular therapeutic agent or class of therapeutic agents. Accordingly, as IBD patients in general, CD and UC patients in particular, seek treatment, there is considerable trial and error involved in the search for therapeutic agent(s) effective for a particular patient. Such trial and error often involves considerable risk and discomfort to the patient to find the most effective therapy. Thus, there is a need for more effective means for determining which patients will respond to which treatment and for incorporating such determinations into more effective treatment regimens for IBD patients.

It would therefore be highly advantageous to have additional diagnostic biomarkers and methods, including predictive diagnostic biomarkers methods, which can be used to objectively identify patients most likely to respond to treatment with various IBD therapeutic agents, including therapeutics that target IL-1β and/or IL-18, such as multispecific anti-IL-1β/anti-IL18 antibodies or combinations of anti-IL-1β antibody and anti-IL-18 antibody. Thus, there is a continuing need to identify new biomarkers associated with ulcerative colitis, Crohn's Disease as well as other inflammatory bowel disorders and that are predictive of therapeutic response. In addition, statistically and biologically significant and reproducible information regarding such associations could be utilized as an integral component in efforts to identify specific subsets of UC or CD patients who would be expected to significantly benefit from treatment with certain therapeutic agents, for example where the therapeutic agent is or has been shown in clinical studies to be of therapeutic benefit in such specific UC or CD patient subpopulation.

The invention described herein meets certain of the above-described needs and provides other benefits.

All references cited herein, including patent applications and publications, are incorporated by reference in their entirety for any purpose.

### SUMMARY

The present invention is based at least in part on the identification of certain genes that are differentially expressed in intestinal tissue of IBD subjects, and in certain cases, in the serum, compared to non-IBD subjects. In addition, the invention is based at least in part on the identification of certain genes that are differentially expressed in fibrotic intestinal tissue, for example, in Crohn's disease subjects, and in certain cases in the serum, compared to non-fibrotic or inflammatory tissue or in the case of serum, compared to non-IBD or non-fibrotic/fibrostenotic IBD subjects.

Accordingly, in one aspect, methods of diagnosing, or aiding in diagnosing, an inflammatory bowel disease are provided. In certain embodiments, the methods comprise (a) measuring in a biological sample obtained from the subject the expression level of one or a combination of genes, or the expression of one or a combination of proteins encoded by the one or the combinations of genes, and (b) comparing the expression level of the one or the combination of genes, or the one or the combination of proteins, measured in (a) to a reference level, wherein the level of the one or the combination of genes, or the level of the one or the combination of proteins encoded by the one or the combination of genes, above the reference level indicates that the subject has an inflammatory bowel disease. In certain embodiments, the methods further comprise providing a diagnosis of inflammatory bowel disease when the level of the one or the combination of genes, or the one or the combination of proteins, measured in (a) is above the reference level. In certain embodiments, the one or the combination of genes, or the one or the combination of proteins encoded by the one or the combination of genes, is selected from CSF3 (GCSF), IL-24, SERPINB3, SERPINB4, AMIGO2, SERPINB7, ABAT, PF4, STEAP2, ELN, CCL4, VEGFA, DACT1, KCNMB4, PDLIM4, TGFBR1, KCNE1L, HIF1A, SLC25A45, OSMR, P4HA2, ELF3, TGIF1, TMEM158, COL7A1, COL16A1, amphiregulin (AREG), and IL-11. In certain embodiments, the one or the combination of genes, or the one or the combination of proteins encoded by the one or the combination of genes, is selected from CSF3 (GCSF), TMEM158, Col7A1, Col16A1, amphiregulin (AREG), IL-11. In certain embodiments, the one or the combination of genes, or the one or the combination of proteins encoded by the one or the combination of genes, is selected from CSF3 (GCSF) and amphiregulin. In certain embodiments, the inflammatory bowel disease is ulcerative colitis or Crohn's disease.

In some embodiments of diagnosing or aiding in the diagnosing of an inflammatory bowel disease, the expression level measured of one or a combination of genes, or the expression of one or a combination of proteins encoded by the one or the combinations of genes, is selected from IL-1β, CASP1, and p20. In certain embodiments, the expression level of one or a combination of IL-1β, CASP1, and p20 above the reference level indicates that the subject has an inflammatory bowel disease. In certain embodiments, the methods further comprise providing a diagnosis of inflammatory bowel disease when the level of the one or the combination of IL-1β, CASP1, and p20 is above the reference level. In certain embodiments, the inflammatory bowel disease is ulcerative colitis or Crohn's disease.

In another aspect, methods of diagnosing, or aiding in diagnosing, fibrotic Crohn's disease or fibrostenotic Crohn's disease are provided. In certain embodiments, the methods comprise (a) measuring in a biological sample obtained from the subject the expression level of one or a combination of genes, or the expression of one or a combination of proteins encoded by the one or the combinations of genes, and (b) comparing the expression level of the one or the combination of genes, or the one or the combination of proteins, measured in (a) to a reference level, wherein the level of the one or the combination of genes, or the level of the one or the combination of proteins encoded by the one or the combination of genes, above the reference level indicates that the subject has an inflammatory bowel disease. In certain embodiments, the methods further comprise providing a diagnosis of inflammatory bowel disease when the level of the one or the combination of genes, or the one or the combination of proteins, measured in (a) is above the reference level. In certain embodiments, the one or the combination of genes, or the one or the combination of proteins encoded by the one or the combination of genes, is selected from COL7A1, COL16A1, amphiregulin, IL-11, AEBP1, and IL1R1. In certain embodiments, the one or the combination of genes, or the one or the combination of proteins encoded by the one or the combination of genes, is selected from MMP3, INHBA, COL5A2, CHN1, LMCD1, COL12A1, COL7A1, COL18A1, TMEM158, FAM65C, IGFBP5, THY1, TMEM132A, PXDN, GPR68, TWIST1, COL4A1, SERPINH1, AEBP1, NAB2, TMEM45A, TMEM121, VIM, NOTCH4, and TIMP2. In certain embodiments, the reference level is obtained by measuring the expression level of the same one or combination of genes, or the same one or combination of proteins, in nonfibrotic tissue.

In certain of the above embodiments, the biological sample is intestinal tissue. In certain of the above embodiments, the expression of the one or the combination of genes is measured using a PCR method or a microarray chip. In certain of the above embodiments, the expression of the one or the combination of proteins is measured using an immunoassay or an immunohistochemical assay. In some embodiments, the immunoassay is an ELISA assay. In some of the above embodiments, the reference level is obtained by measuring the expression level of the same one or combination of genes, or the same one or combination of proteins, in a biological sample obtained from a subject who does not have an inflammatory bowel disorder.

In yet another aspect, methods of diagnosing, or aiding in diagnosing, a subtype of Crohn's disease in a subject are provided. In certain embodiments, the methods comprise (a) measuring in a biological sample obtained from intestinal tissue of the subject the expression level of IL18 and (b) comparing the expression level of IL18 measured in (a) to a reference level, wherein a level of IL18 above the reference level indicates that the subject has a subtype of Crohn's disease wherein the subtype is fibrotic Crohn's disease or fibrostenotic Crohn's disease. In certain embodiments, the methods further comprise providing a diagnosis of fibrotic Crohn's disease or fibrostenotic Crohn's disease when the level of IL18 measured in (a) is above the reference level. In certain embodiments, the reference level is obtained by measuring the expression level of IL18 in a biological sample obtained from intestinal tissue of a subject who does not have an inflammatory bowel disorder. In certain embodiments, the reference level is obtained by measuring the expression level of IL18 in a biological sample obtained from intestinal tissue of a subject who has inflammatory Crohn's disease. In certain embodiments, the reference level is obtained by measuring the expression level of IL18 in a biological sample obtained from intestinal tissue of a subject who has ulcerative colitis. In certain embodiments, the expression level is measured using an immunoassay. In certain embodiments, the immunoassay is an ELISA assay.

In another aspect, further additional methods of diagnosing, or aiding in diagnosing, an inflammatory bowel disease in a subject are provided. In certain embodiments, the methods comprise (a) measuring in a serum sample obtained from the subject the expression level of GCSF and (b) comparing the expression level of GCSF measured in (a) to a reference level, wherein a level of GCSF in the serum sample of the subject above the reference level indicates the subject has an inflammatory bowel disease. In certain embodiments, the methods further comprise providing a diagnosis of inflammatory bowel disease when the level of GCSF measured in (a) is above the reference level. In certain embodiments, the inflammatory bowel disease is ulcerative colitis or inflammatory Crohn's disease, or fibrotic Crohn's disease or fibrostenotic Crohn's disease. In certain embodiments, the expression level of GCSF is measured using an immunoassay. In certain embodiments, the immunoassay is an ELISA assay. In certain embodiments, the reference level is obtained by measuring the expression level of GCSF in a serum sample obtained from a subject who does not have an inflammatory bowel disorder.

In one aspect, methods of treating inflammatory bowel disease in a patient are provided. In certain embodiments, the methods comprise (a) measuring in a biological sample obtained from the patient the expression level of one or a combination of genes, or the expression of one or a combination of proteins encoded by the one or the combinations of genes, selected from CSF3 (GCSF), IL-24, SERPINB3, SERPINB4, AMIGO2, SERPINB7, ABAT, PF4, STEAP2, ELN, CCL4, VEGFA, DACT1, KCNMB4, PDLIM4, TGFBR1, KCNE1L, HIF1A, SLC25A45, OSMR, P4HA2, ELF3, TGIF1, TMEM158, COL7A1, COL16A1, amphiregulin (AREG), and IL-11, (b) comparing the expression level of the one or the combination of genes, or the one or the combination of proteins, measured in (a) to a reference level; and (c) administering an anti-IL-1β antibody, an anti-IL-18 antibody, or a multispecific anti-IL-1β/anti-IL-18 antibody to the patient when the level of the one or the combination of genes, or the one or the combination of proteins, measured in (a) is above the reference level. In certain embodiments, the one or the combination of genes, or the one or the combination of proteins encoded by the one or the combination of genes, is selected from CSF3 (GCSF), TMEM158, Col7A1, Col16A1, amphiregulin (AREG), IL-11. In certain embodiments, the one or the combination of genes, or the one or the combination of proteins encoded by the one or the combination of genes, is selected from CSF3 (GCSF) and amphiregulin. In certain embodiments, the one or the combination of genes, or the one or the combination of proteins encoded by the one or the combination of genes, is selected from MMP3, INHBA, COL5A2, CHN1, LMCD1, COL12A1, COL7A1, COL18A1, TMEM158, FAM65C, IGFBP5, THY1, TMEM132A, PXDN, GPR68, TWIST1, COL4A1, SERPINH1, AEBP1, NAB2, TMEM45A, TMEM121, VIM, NOTCH4, AEBP1, IL1R1, and TIMP2. In certain embodiments, the one or the combination of genes, or the one or the combination of proteins encoded by the one or the combination of genes, is selected from IL-1β, CASP1, and p20. In certain embodiments, an anti-IL-1β antibody, an anti-IL-18 antibody, or a multispecific anti-IL-1β/anti-IL-18 antibody is provided for use in treating a patient having an inflammatory bowel disease, wherein the patient is treated when the level of any one of the above biomarkers in a sample obtained from the patient is above the reference level. In certain embodiments, the inflammatory bowel disease is ulcerative colitis, or inflammatory Crohn's disease, or fibrotic Crohn's disease, or fibrostenotic Crohn's disease. In certain embodiments, the expression level is measured using a PCR method or a microarray chip. In certain embodiments, the expression level is measured using an immunoassay or an ELISA assay.

In another aspect, additional methods of treating inflammatory bowel disease in a patient are provided. In certain embodiments, the methods comprise (a) measuring in a biological sample obtained from intestinal tissue of the patient the expression level of IL18; (b) comparing the expression level of IL18 measured in (a) to a reference level; and (c) administering an anti-IL-1β antibody, an anti-IL-18 antibody, or a multispecific anti-IL-1β/anti-IL-18 antibody to the patient when the level of the one or the combination of genes, or the one or the combination of proteins, measured in (a) is above the reference level. In certain embodiments, an anti-IL-1β antibody, an anti-IL-18 antibody, or a multispecific anti-IL-1β/anti-IL-18 antibody is provided for use in treating a patient having an inflammatory bowel disease, wherein the patient is treated when the level of IL18 in an intestinal tissue sample of the patient is above the reference level. In certain embodiments, the inflammatory bowel disease is ulcerative colitis, or inflammatory Crohn's disease, or fibrotic Crohn's disease, or fibrostenotic Crohn's disease. In certain embodiments, the expression level is measured using a PCR method or a microarray chip.

In yet still another aspect, additional methods of treating an inflammatory bowel disease in a patient are provided. In certain embodiments, the methods comprise (a) measuring in a serum sample obtained from the subject the expression level of GCSF; (b) comparing the expression level of GCSF measured in (a) to a reference level; and (c) administering an anti-IL-1β antibody, an anti-IL-18 antibody, or a multispecific anti-IL-1β/anti-IL-18 antibody to the patient when the level of GCSF measured in (a) is above the reference level. In certain embodiments, an anti-IL-1β antibody, an anti-IL-18 antibody, or a multispecific anti-IL-1β/anti-IL-18 antibody is provided for use in treating a patient having an inflammatory bowel disease, wherein the patient is treated when the level of GCSF in a serum sample of the patient is above the reference level. In certain embodiments, the inflammatory bowel disease is ulcerative colitis, or inflammatory Crohn's disease, or fibrotic Crohn's disease, or fibrostenotic Crohn's disease. In certain embodiments, the expression level is measured using an immunoassay or an ELISA assay.

### BRIEF DESCRIPTION OF THE DRAWINGS

FIGS. 1A, 1B and 1C show increased levels of IL-1β in resected tissue obtained from IBD patients or from non-IBD patients as described in Example 1. FIG. 1A: Comparison of IL-1β mRNA levels between CD patients, UC patients and non-IBD patients, disease status is indicated on the horizontal axis and relative mRNA expression level is indicated on the vertical axis; FIG. 1B: comparison of IL-1β mRNA levels in CD patients undergoing bowel resection for fibrotic or inflammatory disease or from non-IBD patients (as indicated on the horizontal axis); relative mRNA expression level as indicated on the vertical axis. All mRNA levels were normalized to the level of the housekeeping gene, GAPDH. FIG. 1C: Immunohistochemistry staining for IL-1β protein; upper panel shows a section of CD patient resected tissue at low magnification indicating an area of greater staining than surrounding tissue; lower panel shows a higher power view of the same section as indicated by the arrow.

FIG. 2 shows immunoblot analysis of non-IBD, UC or CD biopsy tissues as described in Example 2.

FIGS. 3A, 3B, 3C, 3D, 3E and 3F show mRNA levels in three primary subepithelial myofibroblast cell lines as determined by qRT-PCR following stimulation with media, IL-1β, or TNFα as described in Example 1. In each case, resultant data was normalized to GAPDH. Cells were stimulated for 24 hr except in panel (3E) where they were stimulated for 6 hr. FIG. 3A: GCSF; FIG. 3B: TMEM158; FIG. 3C: Col7A1; FIG. 3D: Col16A1; FIG. 3E: amphiregulin (AREG); FIG. 3F: IL11.

FIGS. 4A and 4B show cell supernatant ELISA results as follows: FIG. 4A: GCSF; and FIG. 4B: amphiregulin, following treatment of three primary subepithelial myofibroblast cell lines treated for 24 hours with media, IL-1β, or TNFα as described in Example 1.

FIGS. 5A and 5B. FIG. 5A shows serum GCSF levels in patients with inflammatory CD (iCD), fibrotic/fibrostenotic CD (fCD), UC, colon cancer, diverticulitis, or healthy controls, as indicated; LLOD=lower limit of detection, and FIG. 5B shows the correlation between IL-1β levels in colon lysate and serum GCSF as described in Example 1.

FIGS. 6A, 6B, 6C, 6D, 6E, and 6F show the level of RNA expression as determined by qRT-PCR for the indicated collagen and fibrosis-associated genes in CD tissue sections matched for RNA isolation, H&E staining (which yielded an inflammation score), and SMA staining by IHC as described in Example 1. Sections were scored by a pathologist for SMA; SMA+ samples are considered to have evidence of fibrosis. FIG. 6A: Collagen subtype 7A1; FIG. 6B: collagen subtype 16A1; FIG. 6C: amphiregulin; FIG. 6D: IL-11 FIG. 6E: AEBP1; FIG. 6F: IL1R1.

FIGS. 7A, 7B, 7C, 7D, 7E, and 7F show IL18 and IL18BP protein levels in intestinal tissue lysate and in serum from matched patient samples as described in Example 2. FIG. 7A: Intestinal tissue lysate IL18; FIG. 7B: serum IL18; FIG. 7C: intestinal tissue lysate IL18BP; FIG. 7D: serum IL18BP; FIG. 7E: intestinal tissue lysate IL18:IL18BP ratio; FIG. 7F: comparision of intestinal tissue lysate IL18 versus serum IL18BP for inflammatory CD (stippled circles) and fibrotic CD (open circles with dotted border).

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### Certain Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

Ranges provided in the specification and appended claims include both end points and all points between the end points. Thus, for example, a range of 2.0 to 3.0 includes 2.0, 3.0, and all points between 2.0 and 3.0.

“Treatment,” “treating,” and grammatical variations thereof refer to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

“Treatment regimen” refers to a combination of dosage, frequency of administration, or duration of treatment, with or without addition of a second medication.

“Effective treatment regimen” refers to a treatment regimen that will offer beneficial response to a patient receiving the treatment.

“Patient response” or “patient responsiveness” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e., reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment. The term “responsiveness” refers to a measurable response, including complete response (CR) and partial response (PR).

As used herein, “complete response” or “CR” means the disappearance of all signs of inflammation or remission in response to treatment. This does not necessarily mean the disease has been cured.

“Partial response” or “PR” refers to a decrease of at least 50% in the severity of inflammation, in response to treatment.

A “beneficial response” of a patient to treatment with a therapeutic agent and similar wording refers to the clinical or therapeutic benefit imparted to a patient at risk for or suffering from a gastrointestinal inflammatory disorder from or as a result of the treatment with the agent. Such benefit includes cellular or biological responses, a complete response, a partial response, a stable disease (without progression or relapse), or a response with a later relapse of the patient from or as a result of the treatment with the agent.

As used herein, “non-response” or “lack of response” or similar wording means an absence of a complete response, a partial response, or a beneficial response to treatment with a therapeutic agent.

“A patient maintains responsiveness to a treatment” when the patient′ responsiveness does not decrease with time during the course of a treatment.

The term “sample,” or “test sample” as used herein, refers to a composition that is obtained or derived from a subject of interest that contains a cellular and/or other molecular entity that is to be characterized and/or identified, for example based on physical, biochemical, chemical and/or physiological characteristics. In one embodiment, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids; and cells from any time in gestation or development of the subject or plasma. The term “sample,” or “test sample” includes biological samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g. a thin slice of tissue or cells cut from a tissue sample. Samples include, but are not limited to, whole blood, blood-derived cells, serum, plasma, lymph fluid, synovial fluid, cellular extracts, and combinations thereof. In one embodiment, the sample is a clinical sample. In another embodiment, the sample is used in a diagnostic assay.

A “reference sample,” as used herein, refers to any sample, standard, or level that is used for comparison purposes. In one embodiment, a reference sample is obtained from a healthy and/or non-diseased part of the body (e.g., tissue or cells) of the same subject or patient. In another embodiment, a reference sample is obtained from an untreated tissue and/or cell of the body of the same subject or patient. In yet another embodiment, a reference sample is obtained from a healthy and/or non-diseased part of the body (e.g., tissues or cells) of an individual who is not the subject or patient. In even another embodiment, a reference sample is obtained from an untreated tissue and/or cell part of the body of an individual who is not the subject or patient.

“Gastrointestinal inflammatory disorders” are a group of chronic disorders that cause inflammation and/or ulceration in the mucous membrane. These disorders include, for example, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis.

“Inflammatory Bowel Disease” or “IBD” is used interchangeably herein to refer to diseases of the bowel that cause inflammation and/or ulceration and includes without limitation Crohn's disease and ulcerative colitis.

“Crohn's disease (CD)” and “ulcerative colitis (UC)” are chronic inflammatory bowel diseases of unknown etiology. Crohn's disease, unlike ulcerative colitis, can affect any part of the bowel. The most prominent feature Crohn's disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of Crohn's disease may be continuous or relapsing, mild or severe, but unlike ulcerative colitis, Crohn's disease is not curable by resection of the involved segment of bowel. Most patients with Crohn's disease require surgery at some point, but subsequent relapse is common and continuous medical treatment is usual.

Crohn's disease may involve any part of the alimentary tract from the mouth to the anus, although typically it appears in the ileocolic, small-intestinal or colonic-anorectal regions. Histopathologically, the disease manifests by discontinuous granulomatomas, crypt abscesses, fissures and aphthous ulcers. The inflammatory infiltrate is mixed, consisting of lymphocytes (both T and B cells), plasma cells, macrophages, and neutrophils. There is a disproportionate increase in IgM- and IgG-secreting plasma cells, macrophages and neutrophils.

Anti-inflammatory drugs sulfasalazine and 5-aminosalisylic acid (5-ASA) are used for treating mildly active colonic Crohn's disease and are commonly prescribed in an attempt to maintain remission of the disease. Metroidazole and ciprofloxacin are similar in efficacy to sulfasalazine and are particularly prescribed for treating perianal disease. In more severe cases, corticosteroids are prescribed to treat active exacerbations and can sometimes maintain remission. Azathioprine and 6-mercaptopurine have also been used in patients who require chronic administration of corticosteroids. It has been suggested that these drugs may play a role in the long-term prophylaxis. Unfortunately, there can be a very long delay (up to six months) before onset of action in some patients. Antidiarrheal drugs can also provide symptomatic relief in some patients. Nutritional therapy or elemental diet can improve the nutritional status of patients and induce symptomatic improvement of acute disease, but it does not induce sustained clinical remissions. Antibiotics are used in treating secondary small bowel bacterial overgrowth and in treatment of pyogenic complications.

“Ulcerative colitis (UC)” afflicts the large intestine. The course of the disease may be continuous or relapsing, mild or severe. The earliest lesion is an inflammatory infiltration with abscess formation at the base of the crypts of Lieberkuhn. Coalescence of these distended and ruptured crypts tends to separate the overlying mucosa from its blood supply, leading to ulceration. Symptoms of the disease include cramping, lower abdominal pain, rectal bleeding, and frequent, loose discharges consisting mainly of blood, pus and mucus with scanty fecal particles. A total colectomy may be required for acute, severe or chronic, unremitting ulcerative colitis.

The clinical features of UC are highly variable, and the onset may be insidious or abrupt, and may include diarrhea, tenesmus and relapsing rectal bleeding. With fulminant involvement of the entire colon, toxic megacolon, a life-threatening emergency, may occur. Extraintestinal manifestations include arthritis, pyoderma gangrenoum, uveitis, and erythema nodosum.

Treatment for UC includes sulfasalazine and related salicylate-containing drugs for mild cases and corticosteroid drugs in severe cases. Topical administration of either salicylates or corticosteroids is sometimes effective, particularly when the disease is limited to the distal bowel, and is associated with decreased side effects compared with systemic use. Supportive measures such as administration of iron and antidiarrheal agents are sometimes indicated. Azathioprine, 6-mercaptopurine and methotrexate are sometimes also prescribed for use in refractory corticosteroid-dependent cases.

An “effective dosage” refers to an amount effective, at dosages and for periods of time necessary, to achieve the desired therapeutic or prophylactic result.

As used herein, the term “patient” refers to any single subject for which treatment is desired. In certain embodiments, the patient herein is a human.

A “subject” herein is typically a human. In certain embodiments, a subject is a non-human mammal. Exemplary non-human mammals include laboratory, domestic, pet, sport, and stock animals, e.g., mice, cats, dogs, horses, and cows. Typically, the subject is eligible for treatment, e.g., treatment of a gastrointestinal inflammatory disorder.

The terms “antibody” and “immunoglobulin” are used interchangeably in the broadest sense and include monoclonal antibodies (for example, full length or intact monoclonal antibodies), polyclonal antibodies, multivalent antibodies, multi specific antibodies (e.g., bispecific, trispecific etc. antibodies so long as they exhibit the desired biological activity) and may also include certain antibody fragments (as described in greater detail herein). An antibody can be human, humanized and/or affinity matured.

“Antibody fragments” comprise only a portion of an intact antibody, where in certain embodiments, the portion retains at least one, and typically most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 55-93 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody as determined by the Lowry method, and often more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The term “hypervariable region,” “HVR,” or “HV,” when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these HVRs are noted below.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat |
| | | | | | Number- |
| | | | | | ing) |
| H1 | H31-H35 | H26-H35 | H26-H32 | H30-H35 | (Chothia |
| | | | | | Number- |
| | | | | | ing) |
| H2 | H50-H65 | H50-H58 | H53-H55 | H47-H58 | |
| H3 | H95-H102 | H95-H102 | H96-H101 | H93-H101 | |

Hypervariable regions may comprise “extended hypervariable regions” as follows: 24-36 or 24-34 (L1), 46-56 or 49-56 or 50-56 or 52-56 (L2) and 89-97 (L3) in the VL and 26-35 (H1), 50-65 or 49-65 (H2) and 93-102, 94-102 or 95-102 (H3) in the VH. The variable domain residues are numbered according to Kabat et al., supra for each of these definitions.

“Framework” or “FR” residues are those variable domain residues other than the hypervariable region residues as herein defined.

A “human consensus framework” is a framework which represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. In one embodiment, for the VL, the subgroup is subgroup kappa I as in Kabat et al. In one embodiment, for the VH, the subgroup is subgroup III as in Kabat et al.

An “affinity matured” antibody is one with one or more alterations in one or more CDRs thereof which result in an improvement in the affinity of the antibody for antigen, compared to a parent antibody which does not possess those alteration(s). In certain embodiments, affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1996); Yelton et al. J. Immunol. 155:1994-2004 (1995); Jackson et al., J. Immunol. 154(7):3310-9 (1995); and Hawkins et al. J. Mol. Biol. 226:889-896 (1992).

The phrase “substantially similar,” or “substantially the same,” as used herein, denotes a sufficiently high degree of similarity between two numeric values such that one of skill in the art would consider the difference between the two values to be of little or no biological and/or statistical significance within the context of the biological characteristic measured by said values.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art.

The term “variable” in connection with antibodies or immunoglobulins refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab=fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known and described generally in, for example, Abbas et al. Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In certain embodiments, the variant Fc region has at least one amino acid substitution compared to a native sequence Fc region or to the Fc region of a parent polypeptide, e.g., from about one to about ten amino acid substitutions, and in certain embodiments from about one to about five amino acid substitutions in a native sequence Fc region or in the Fc region of the parent polypeptide. In certain embodiments, the variant Fc region herein will possess at least about 80% homology with a native sequence Fc region and/or with an Fc region of a parent polypeptide, or at least about 90% homology therewith, or at least about 95% homology therewith.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ respectively. The subunit structures and three-dimensional configurations of different classes of immunoglobulins are well known.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Human effector cells” are leukocytes which express one or more FcRs and perform effector functions. In certain embodiments, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source thereof, e.g., from blood or PBMCs as described herein.

The terms “Fc receptor” or “FcR” are used to describe a receptor that binds to the Fc region of an antibody. In certain embodiments, FcR is a native sequence human FcR. Moreover, FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)), and regulates homeostasis of immunoglobulins. Antibodies with improved binding to the neonatal Fc receptor (FcRn), and increased half-lives, are described in WO00/42072 (Presta, L.) and US2005/0014934A1 (Hinton et al.). These antibodies comprise an Fc region with one or more substitutions therein which improve binding of the Fc region to FcRn. For example, the Fc region may have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). In certain embodiments, the Fc region-comprising antibody variant with improved FcRn binding comprises amino acid substitutions at one, two or three of positions 307, 380 and 434 of the Fc region thereof (Eu numbering of residues).

“Single-chain Fv” or “scFv” antibody fragments comprise the VH and VL domains of antibody, wherein these domains are present in a single polypeptide chain. In certain embodiments, the Fv polypeptide further comprises a polypeptide linker between the VH and VL domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “affinity matured” antibody is one with one or more alterations in one or more hypervariable regions thereof which result an improvement in the affinity of the antibody for antigen, compared to a parent antibody which does not possess those alteration(s). In certain embodiments, affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. In certain embodiments, amino acid sequence variants will possess at least about 70% homology with the main species antibody, or they will be at least about 80%, or at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g., deamidated antibody variant), a basic variant, an antibody with an amino-terminal leader extension (e.g. VHS-) on one or two light chains thereof, an antibody with a C-terminal lysine residue on one or two heavy chains thereof, and the like, and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieties attached thereto which differ from one or more carbohydrate moieties attached to a main species antibody. Examples of glycosylation variants herein include antibody with a G1 or G2 oligosaccharide structure, instead a G0 oligosaccharide structure, attached to an Fc region thereof, antibody with one or two carbohydrate moieties attached to one or two light chains thereof, antibody with no carbohydrate attached to one or two heavy chains of the antibody, and the like, and combinations of glycosylation alterations. Where the antibody has an Fc region, an oligosaccharide structure may be attached to one or two heavy chains of the antibody, e.g. at residue 299 (298, Eu numbering of residues).

The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-1β, IL-2, IL-3, IL-4, IL-5, IL-6, IL-7, IL-8, IL-9, IL-10, IL-11, IL-12, IL-18; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “immunosuppressive agent” as used herein for adjunct therapy refers to substances that act to suppress or mask the immune system of the subject being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir; tacrolimus; glucocorticoids such as cortisol or aldosterone; anti-inflammatory agents such as a cyclooxygenase inhibitor; a 5-lipoxygenase inhibitor; or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporine; 6 mercaptopurine; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies or antagonists including anti-interferon-alpha, -beta, or -gamma antibodies, anti-tumor necrosis factor (TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodomase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF or BR3 antibodies or immunoadhesins and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand. (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9.

The term “ameliorates” or “amelioration” as used herein refers to a decrease, reduction or elimination of a condition, disease, disorder, or phenotype, including an abnormality or symptom.

A “symptom” of a disease or disorder (e.g., inflammatory bowel disease, e.g., ulcerative colitis or Crohn's disease) is any morbid phenomenon or departure from the normal in structure, function, or sensation, experienced by a subject and indicative of disease.

The expression “therapeutically effective amount” refers to an amount that is effective for preventing, ameliorating, or treating a disease or disorder (e.g., inflammatory bowel disease, e.g., ulcerative colitis or Crohn's disease). For example, a “therapeutically effective amount” of an antibody refers to an amount of the antibody that is effective for preventing, ameliorating, or treating the specified disease or disorder. Similarly, a “therapeutically effective amount” of a combination of an antibody and a second compound refers to an amount of the antibody and an amount of the second compound that, in combination, is effective for preventing, ameliorating, or treating the specified disease or disorder.

It is to be understood that the terminology “a combination of” two compounds does not mean that the compounds have to be administered in admixture with each other. Thus, treatment with or use of such a combination encompasses a mixture of the compounds or separate administration of the compounds, and includes administration on the same day or different days. Thus the terminology “combination” means two or more compounds are used for the treatment, either individually or in admixture with each other. When an antibody and a second compound, for example, are administered in combination to a subject, the antibody is present in the subject at a time when the second compound is also present in the subject, whether the antibody and second compound are administered individually or in admixture to the subject. In certain embodiments, a compound other than the antibody is administered prior to the antibody. In certain embodiments, a compound other than the antibody is administered after the antibody.

For the purposes herein, “tumor necrosis factor-alpha (TNF-alpha)” refers to a human TNF-alpha molecule comprising the amino acid sequence as described in Pennica et al., Nature, 312:721 (1984) or Aggarwal et al., JBC, 260:2345 (1985).

A “TNF-alpha inhibitor” herein is an agent that inhibits, to some extent, a biological function of TNF-alpha, generally through binding to TNF-alpha and neutralizing its activity. Examples of TNF inhibitors specifically contemplated herein are etanercept (ENBREL®), infliximab (REMICADE®), adalimumab (HUMIRA®), golimumab (SIMPONI™), and certolizumab pegol (CIMZIA®).

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone triamcinolone, and betamethasone.

An “antagonist” refers to a molecule capable of neutralizing, blocking, inhibiting, abrogating, reducing or interfering with the activities of a particular or specified protein, including its binding to one or more receptors in the case of a ligand or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of the protein, and fusion proteins, receptor molecules and derivatives which bind specifically to the protein thereby sequestering its binding to its target, antagonist variants of the protein, antisense molecules directed to the protein, RNA aptamers, and ribozymes against the protein.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and allowing the polymerization of a complementary nucleic acid, generally by providing a free 3′-OH group.

The term “amplification” refers to the process of producing one or more copies of a reference nucleic acid sequence or its complement. Amplification may be linear or exponential (e.g., PCR). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “detection” includes any means of detecting, including direct and indirect detection.

“Elevated expression” or “elevated levels” refers to an increased expression of a mRNA or a protein in a patient relative to a control, such as an individual or individuals who are not suffering from an autoimmune disease, e.g., IBD, or relative to a pre-established threshold or cut-off value, or relative to the median for a population of patients and/or subjects.

“Low expression” or “low expression levels” refers to a decreased expression of a mRNA or a protein in a patient relative to a control, such as an individual or individuals who are not suffering from an autoimmune disease, e.g., IBD, or relative to a pre-established threshold or cut-off value, or relative to the median for a population of patients and/or subjects.

The term “multiplex-PCR” refers to a single PCR reaction carried out on nucleic acid obtained from a single source (e.g., a patient) using more than one primer set for the purpose of amplifying two or more DNA sequences in a single reaction.

The term “biomarker” as used herein refers to an indicator of a phenotype of a patient, e.g, a pathological state or likely responsiveness to a therapeutic agent, which can be detected in a biological sample of the patient. Biomarkers include, but are not limited to, DNA, RNA, protein, carbohydrate, or glycolipid-based molecular markers.

The term “diagnosis” is used herein to refer to the identification or classification of a molecular or pathological state, disease or condition. For example, “diagnosis” may refer to identification of a particular type of IBD, e.g., UC or Crohn's disease. “Diagnosis” may also refer to the classification of a particular subtype of IBD, e.g., by histopathological criteria or by molecular features (e.g., a subtype characterized by expression of one or a combination of particular genes or proteins encoded by said genes).

The term “aiding diagnosis” is used herein to refer to methods that assist in making a clinical determination regarding the presence, or nature, of a particular type of symptom or condition. For example, a method of aiding diagnosis of IBD can comprise measuring the expression of certain genes in a biological sample from an individual.

The phrase “providing a diagnosis” as used herein refers to using the information or data generated relating to the level or presence of any one or more or combination of biomarkers as described herein in a sample of a patient to diagnose inflammatory bowel disease, including ulcerative colitis, Crohn's disease, inflammatory Crohn's disease, fibrotic/fibrostenotic Crohn's disease, in the patient. The information or data may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the level of any one or more or combination of biomarkers as described herein to a reference level. In some embodiments, the information or data includes an indication that any one or more or combination of biomarkers as described herein is present or absent in the sample. In some embodiments, the information or data includes an indication that the patient is diagnosed with inflammatory bowel disease. In some embodiments, the information or data includes an indication that the patient is diagnosed with ulcerative colitis, Crohn's disease, inflammatory Crohn's disease, or fibrotic/fibrostenotic Crohn's disease.

The phrase “recommending a treatment” as used herein refers to using the information or data generated relating to the level or presence of any one or more or combination of biomarkers as described herein in a sample of a patient to identify the patient as suitably treated or not suitably treated with a therapy. In some embodiment the therapy may comprise an anti-IL-1β antibody and/or anti-IL-18 antibody/antibodies. The information or data may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the level of any one or more or combination of biomarkers as described herein to a reference level. In some embodiments, the information or data includes an indication that any one or more or combination of biomarkers as described herein is present or absent in the sample. In some embodiments, the information or data includes an indication that the patient is suitably treated or not suitably treated with a therapy comprising an anti-IL-1β antibody and/or anti-IL-18 antibody/antibodies.

The term “prognosis” is used herein to refer to the prediction of the likelihood of autoimmune disorder-attributable disease symptoms of an autoimmune disease such as IBD.

The term “prediction” is used herein to refer to the likelihood that a patient will respond either favorably or unfavorably to a drug (therapeutic agent) or set of drugs or a therapeutic regimen. In one embodiment, the prediction relates to the extent of those responses. In one embodiment, the prediction relates to whether and/or the probability that a patient will survive or improve following treatment, for example treatment with a particular therapeutic agent, or for a certain period of time without disease recurrence. The predictive methods as described herein can be used clinically to make treatment decisions by choosing the most appropriate treatment modalities for any particular patient. The predictive methods as described herein are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient or remission or sustained remission, following a therapeutic regimen is likely.

A “control subject” refers to a healthy subject who has not been diagnosed as having a particular disease, e.g., IBD, and who does not suffer from any sign or symptom associated with that disease.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments and the like.

A “kit” is any manufacture (e.g a package or container) comprising at least one reagent, e.g., a medicament for treatment of an IBD, e.g., UC or Crohn's Disease, or a probe for specifically detecting, for example, a biomarker gene or protein. In certain embodiments, the manufacture is promoted, distributed, or sold as a unit for performing the methods of the present invention.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

The term “serum sample” refers to any serum sample obtained from an individual. Methods for obtaining sera from mammals are well known in the art.

The term “whole blood” refers to any whole blood sample obtained from an individual. Typically, whole blood contains all of the blood components, e.g., cellular components and plasma. Methods for obtaining whole blood from mammals are well known in the art.

The expression “not responsive to,” “non-response” and grammatical variants thereof, as it relates to the reaction of subjects or patients to one or more of the medicaments (therapeutic agents) that were previously administered to them, describes those subjects or patients who, upon administration of such medicament(s), did not exhibit any or adequate signs of treatment of the disorder for which they were being treated, or they exhibited a clinically unacceptably high degree of toxicity to the medicament(s), or they did not maintain the signs of treatment after first being administered such medicament(s), with the word treatment being used in this context as defined herein. The phrase “not responsive” includes a description of those subjects who are resistant and/or refractory to the previously administered medication(s), and includes the situations in which a subject or patient has progressed while receiving the medicament(s) that he or she is being given, and in which a subject or patient has progressed within 12 months (for example, within six months) after completing a regimen involving the medicament(s) to which he or she is no longer responsive. The non-responsiveness to one or more medicaments thus includes subjects who continue to have active disease following previous or current treatment therewith. For instance, a patient may have active disease activity after about one to three months, or three to six months, or six to 12 months, of therapy with the medicament(s) to which they are non-responsive. Such responsiveness may be assessed by a clinician skilled in treating the disorder in question.

For purposes of non-response to medicament(s), a subject who experiences “a clinically unacceptably high level of toxicity” from previous or current treatment with one or more medicaments experiences one or more negative side-effects or adverse events associated therewith that are considered by an experienced clinician to be significant, such as, for example, serious infections, congestive heart failure, demyelination (leading to multiple sclerosis), significant hypersensitivity, neuropathological events, high degrees of autoimmunity, a cancer such as endometrial cancer, non-Hodgkin's lymphoma, breast cancer, prostate cancer, lung cancer, ovarian cancer, or melanoma, tuberculosis (TB), and the like.

The “amount” or “level” of a biomarker associated with an increased clinical benefit to a patient suffering from a certain disease or disorder, or predictive of response to a particular therapeutic agent or treatment regimen, is a detectable level in a biological sample. These can be measured by methods known to one skilled in the art and also disclosed herein. The expression level or amount of biomarker assessed can be used to determine the response or the predicted response to a treatment or therapeutic agent.

The terms “level of expression” or “expression level” in general are used interchangeably and generally refer to the amount of a polynucleotide or an amino acid product or protein in a biological sample. “Expression” generally refers to the process by which gene-encoded information is converted into the structures present and operating in the cell. Therefore, as used herein, “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

The phrase “an anti-IL-1β antibody and/or anti-IL-18 antibody/antibodies” refers, depending on the context, to (1) an anti-IL-1β antibody, or (2) an anti-IL-18 antibody, or (3) a combination of an anti-IL-1β antibody and an anti-IL-18 antibody (i.e., two antibodies), or (4) an antibody that binds to both IL-1β and IL-18.

The terms “anti-IL-1β antibody” and “an antibody that binds to IL-1β” refer to an antibody that is capable of binding IL-1β with sufficient affinity such that the antibody is useful as a diagnostic and/or therapeutic agent in targeting IL-1β. In one embodiment, the extent of binding of an anti-IL-1β antibody to an unrelated, non-IL-1β protein is less than about 10% of the binding of the antibody to IL-1β as measured, e.g., by a radioimmunoassay (RIA). In certain embodiments, an anti-IL-1β antibody binds to an epitope of IL-1β that is conserved among IL-1β from different species.

The terms “anti-IL-18 antibody” and “an antibody that binds to IL-18” refer to an antibody that is capable of binding IL-18 with sufficient affinity such that the antibody is useful as a diagnostic and/or therapeutic agent in targeting IL-18. In one embodiment, the extent of binding of an anti-IL-18 antibody to an unrelated, non-IL-18 protein is less than about 10% of the binding of the antibody to IL-18 as measured, e.g., by a radioimmunoassay (RIA). In certain embodiments, an anti-IL-18 antibody binds to an epitope of IL-18 that is conserved among IL-18 from different species.

An “inflammasome-mediated disease” refers to any disease where IL-1β and/or IL-18 are elevated relative to normal, uninflamed tissue. Generally, in an inflammasome-mediated disease, caspase-1 processing and/or activation is involved/elevated relative to uninduced control cells. Caspase-1 activity can be measured using commercially available assay kits, e.g., Caspase 1 Fluorometric Assay Kit ((Cat. No. ab394120; AbCam, Cambridge, Mass.), Caspase-1 Colorimetric Assay (Cat. No. BF14100; R&D Systems), etc.

In general, a disease or condition can be considered an IL-1β related disease or condition if it is associated with elevated levels of IL-1β in bodily fluids or tissue or if cells or tissues taken from the body produce elevated levels of IL-1β in culture. Similarly, a disease or condition can be considered an IL-18 related disease or condition if it is associated with elevated levels of IL-18 in bodily fluids or tissue or if cells or tissues taken from the body produce elevated levels of IL-18 in culture. Thus, an IL-1β/IL-18 related disease or condition is associated with elevated levels of IL-1β and IL-18 in bodily fluids or tissue or if cells or tissues taken from the body produce elevated levels of both cytokines in culture.

Examples of IL-1β related diseases are acute pancreatitis; ALS; cachexia/anorexia, including AIDS-induced cachexia; asthma and other pulmonary diseases; autoimmune vasculitis; CIAS1 Associated Periodic Syndromes (CAPS); Neonatal Onset Multisystem Inflammatory Disorder (NOMID/CINCA), systemic onset juvenile idiopathic arthritis, Stills disease, Muckle-Wells syndrome, chronic fatigue syndrome; Clostridium associated illnesses, including Clostridium-associated diarrhea; coronary conditions and indications, including congestive heart failure, coronary restenosis, myocardial infarction, myocardial dysfunction (e.g., related to sepsis), and coronary artery bypass graft; cancers, such as multiple myeloma and myelogenous (e.g., AML and CIVIL) and other leukemias, as well as tumor metastasis; diabetes (e.g., insulin diabetes); endometriosis; familial Cold Autoinflammatory Syndrome (FCAS); familial Mediterranean fever (FMF); fever; fibromyalgia; glomerulonephritis; graft versus host disease/transplant rejection; hemorrhagic shock; hyperalgesia; inflammatory bowel disease; inflammatory conditions of a joint, including psoriatic arthritis (as well as osteoarthritis and rheumatoid arthritis); inflammatory eye disease, as may be associated with, for example, corneal transplant; ischemia, including cerebral ischemia (e.g., brain injury as a result of trauma, epilepsy, hemorrhage or stroke, each of which may lead to neurodegeneration); Kawasaki's disease; learning impairment; lung diseases (e.g., ARDS); myopathies (e.g., muscle protein metabolism, especially in sepsis); neurotoxicity (e.g., as induced by HIV); osteoporosis; pain, including cancer-related pain; Parkinson's disease; periodontal disease; pre-term labor; psoriasis; reperfusion injury; side effects from radiation therapy; sleep disturbance; temporal mandibular joint disease; tumor necrosis factor receptor-associated periodic fever syndrome (TRAPS); uveitis; or an inflammatory condition resulting from strain, sprain, cartilage damage, trauma, orthopedic surgery, infection or other disease processes.

Interleukin 18 plays an important role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, lupus (e.g., Systemic Lupus Erythematosus, and Lupus Nephritis), Crohn's disease, ulcerative colitis, inflammatory bowel disease, insulin dependent diabetes mellitus, thyroiditis, asthma, allergic diseases, psoriasis, psoriasis type 1, psoriasis type 2, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease1 arteriosclerosis, atopic allergy, autoimmune bullous disease, pemphigus vulgaris, pemphigus foliaceus, pemphigoid, linear IgA disease, autoimmune haemolytic anemia, Coombs positive haemolytic anemia, acquired pernicious anemia, juvenile pernicious anemia, myalgic encephalitis/Royal Free Disease. chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency, common variable hypogammaglobulinemia, dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis, classical autoimmune or lupoid hepatitis, type-2 autoimmune hepatitis, anti-LKM antibody hepatitis, autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopaenia, autoimmune neutropenia, renal disease NOS, glomerulonephritides, microscopic vasulitis of the kidneys, Lyme disease, discoid lupus erythematosus, male infertility idiopathic or NOS, sperm autoimmunity, all subtypes of multiple sclerosis, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopenia, idiopathic thrombocytopenia, autoimmune thyroid disease, hyperthyroidism, goitrous autoimmune hypothyroidism or Hashimoto's disease, atrophic autoimmune hypothyroidism, primary myxoedema, phacogenic uveitis, primary vasculitis, vitiligo, acute liver disease, chronic liver diseases, allergy and asthma, mental disorders, depression, schizophrenia, Th2 Type and Thl Type mediated diseases, Chronic Obstructive Pulmonary Disease (COPD), inflammatory, autoimmune and bone diseases.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The term “knob-into-hole” or “KnH” as mentioned herein refers to the technology directing the selectively pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, C_{L}:C_{H}1 interfaces or V_{H}/V_{L} interfaces of antibodies (e.g., US20007/0178552, WO 96/027011, WO 98/050431 and Zhu et al. (1997) Protein Science 6:781-788). This is especially useful in driving the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. In fact, KnH technology can be used to pair two different receptor extracellular domains together or any other polypeptide sequences that comprises different target recognition sequences (e.g., including affibodies, peptibodies and other Fc fusions).

The term “multispecific antibody” is used in the broadest sense and refers to an antibody that has polyepitopic specificity. Such multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with at least two single variable domains binding to different epitopes, full length antibodies, antibody fragments such as Fab, Fv, dsFv, scFv, diabodies, tandem antibodies, linear antibodies and triabodies, antibody fragments that have been linked covalently or bind to each other through non-covalent interactions. Other examples of antibody formats have been used or may be used to create multispecific antibodies include, but are not limited to, Fc fusions of diabodies, tandem antibodies, and single chain antibodies (e.g, Db-Fc, taDb-Fc, taDb-CH3 and (scFV)4-Fc), knob-N-hole (KnH) antibodies, octopus antibodies and DAF antibodies.

“Multispecific Molecule” as used herein refers to a molecule that has polyepitopic specificity. “Polyepitopic specificity” refers to the ability to specifically bind to at least two different epitopes on one target molecule or on different target molecules (e.g, an anti-IL-1β/IL-18 multispecific antibody). “Monospecific” refers to the ability to bind only one epitope. According to one embodiment a multispecific molecule binds to each epitope with an affinity of 5 μM to 0.001 pM, 3 μM to 0.001 pM, 1 μM to 0.001 pM, 0.5 μM to 0.001 pM or 0.1 μM to 0.001 pM. The term “bispecific” as used herein refers to the ability to bind two epitopes. Examples of molecules that support or can be engineered to support polyepitopic specificity include, but is not limited to, antibodies, affibodies, immunoadhesins, peptibodies and other Fc fusions.

The term “octopus” antibody or antibodies as used herein refers to multivalent antibodies comprising an Fc region and two or more antigen binding sites amino-terminal to the Fc region (e.g., WO01/77342, Wu et al. (2007) Nature Biotechnology, and WO 2007/024715). In one embodiment, the configuration of a polypeptide of the antibody is VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. In one embodiment, X1 or X2 is a CH1 domain, a portion of a CH1 domain, some other linker sequence such as a GS linker or some combination thereof (e.g., page 5 of WO 2007/024715).

A nucleic acid is “operably linked,” as used herein, when it is placed into a functional relationship with another nucleic acid sequence. For example, DNA for a presequence or secretory leader is operably linked to DNA for a antibody if it is expressed as a preprotein that participates in the secretion of the antibody; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, an enhancer may not have to be contiguous. Linking is accomplished by ligation at convenient restriction sites. If such sites do not exist, synthetic oligonucleotide adaptors or linkers are used in accordance with conventional practice.

“Peptibody” or “peptibodies” refers to a fusion of peptide sequences with an Fc domain. See U.S. Pat. No. 6,660,843, issued Dec. 9, 2003 to Feige et al. (incorporated by reference in its entirety). They include one or more peptides linked to the N-terminus, C-terminus, amino acid sidechains, or to more than one of these sites. Peptibody technology enables design of therapeutic agents that incorporate peptides that target one or more ligands or receptors, tumor-homing peptides, membrane-transporting peptides, and the like. Peptibody technology has proven useful in design of a number of such molecules, including linear and disulfide-constrained peptides, “tandem peptide multimers” (i.e., more than one peptide on a single chain of an Fc domain). See, for example, U.S. Pat. No. 6,660,843; U.S. Pat. App. No. 2003/0195156, published Oct. 16, 2003 (corresponding to WO 02/092620, published Nov. 21, 2002); U.S. Pat. App. No. 2003/0176352, published Sep. 18, 2003 (corresponding to WO 03/031589, published Apr. 17, 2003); U.S. Ser. No. 09/422,838, filed Oct. 22, 1999 (corresponding to WO 00/24770, published May 4, 2000); U.S. Pat. App. No. 2003/0229023, published Dec. 11, 2003; WO 03/057134, published Jul. 17, 2003; U.S. Pat. App. No. 2003/0236193, published Dec. 25, 2003 (corresponding to PCT/US04/010989, filed Apr. 8, 2004); U.S. Ser. No. 10/666,480, filed Sep. 18, 2003 (corresponding to WO 04/026329, published Apr. 1, 2004), each of which is hereby incorporated by reference in its entirety.

For the purposes herein, a “pharmaceutical composition” is one that is adapted and suitable for administration to a mammal, especially a human. Thus, the composition can be used to treat a disease or disorder in the mammal. Moreover, the protein in the composition has been subjected to one or more purification or isolation steps, such that contaminant(s) that might interfere with its therapeutic use have been separated therefrom. Generally, the pharmaceutical composition comprises the therapeutic protein and a pharmaceutically acceptable carrier or diluent. The composition is usually sterile and may be lyophilized. Pharmaceutical preparations are described in more detail below.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkage may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C.) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

The term “receptor binding domain” is used to designate any native ligand for a receptor, including cell adhesion molecules, or any region or derivative of such native ligand retaining at least a qualitative receptor binding ability of a corresponding native ligand. This definition, among others, specifically includes binding sequences from ligands for the above-mentioned receptors.

“Secretion signal sequence” or “signal sequence” refers to a nucleic acid sequence encoding a short signal peptide that can be used to direct a newly synthesized protein of interest through a cellular membrane, usually the inner membrane or both inner and outer membranes of prokaryotes. As such, the protein of interest such as the immunoglobulin light or heavy chain polypeptide is secreted into the periplasm of the prokaryotic host cells or into the culture medium. The signal peptide encoded by the secretion signal sequence may be endogenous to the host cells, or they may be exogenous, including signal peptides native to the polypeptide to be expressed. Secretion signal sequences are typically present at the amino terminus of a polypeptide to be expressed, and are typically removed enzymatically between biosynthesis and secretion of the polypeptide from the cytoplasm. Thus, the signal peptide is usually not present in a mature protein product.

The expression “single domain antibodies” (sdAbs) or “single variable domain (SVD) antibodies” generally refers to antibodies in which a single variable domain (V_{H} or V_{L}) can confer antigen binding. In other words, the single variable domain need not interact with another variable domain in order to bind the target antigen. Examples of single domain antibodies include, but is not limited to, those derived from nature such as camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; Febs Lett (1994) 339:285-290; WO00/29004; WO 02/051870).

As used herein, a “therapeutic antibody” is an antibody that is effective in treating a disease or disorder in a mammal with or predisposed to the disease or disorder.

“Target molecule” refers to a molecule that is capable of binding a target recognition site. Examples of target molecule:target recognition site interactions include antigen:antibody variable domain interactions, receptor:ligand interactions, ligand:receptor interactions, adhesin:adhesin interactions, biotin:strepavidin interactions, etc. In one embodiment, the target molecule is a biological molecule.

The term “vector,” as used herein, is intended to refer to a nucleic acid molecule capable of transporting another nucleic acid to which it has been linked. One type of vector is a “plasmid”, a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (for example, bacterial vectors having a bacterial origin of replication and episomal mammalian vectors). Other vectors (for example, non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

An antibody that “selectively binds” a target molecule with significantly better affinity than it binds to other molecules that are not the target molecule. The relative binding and/or binding affinity may be demonstrated in a variety of methods accepted in the art including, but not limited to: enzyme linked immunosorbent assay (ELISA) and fluorescence activated cell sorting (FACS). In some embodiments, as antibody binds a target molecule with at least about 1 log higher concentration reactivity than it binds to a non-target molecule, as determined by an ELISA.

A variety of additional terms are defined or otherwise characterized herein.

### Exemplary Antibodies

Soluble human IL-1β or human IL-18, or fragments thereof, optionally conjugated to other molecules, can be used as immunogens for generating antibodies. Alternatively, or additionally, cells expressing human IL-1β or human IL-18 can be used as the immunogen. Such cells can be derived from a natural source or may be cells that have been transformed by recombinant techniques to express human IL-1β or human IL-18. Other forms of human IL-1β or human IL-18 useful for preparing antibodies will be apparent to those in the art.

a. Polyclonal Antibodies

Polyclonal antibodies are typically raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, for example, 100 μg or 5 μg of the protein or conjugate (for rabbits or mice, respectively) with 3 volumes of Freund's complete adjuvant and injecting the solution intradermally at multiple sites. Approximately one month later, the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Typically, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

b. Monoclonal Antibodies

Monoclonal antibodies may be made using the hybridoma method first described by Kohler et al., 1975, Nature, 256:495, or may be made by recombinant DNA methods (See, for example, U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, 1986, Monoclonal Antibodies: Principles and Practice, pp. 59-103 (Academic Press)).

The hybridoma cells thus prepared are seeded and grown in a suitable culture medium that typically contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

In certain embodiments, myeloma cells are those that fuse efficiently, support stable high-level production of antibody by the selected antibody-producing cells, and are sensitive to a medium such as HAT medium. Among these, exemplary myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, 1984, J Immunol., 133:3001; Brodeur et al., 1987, Monoclonal Antibody Production Techniques and Applications, pp. 51-63 (Marcel Dekker, Inc., New York)).

Culture medium in which hybridoma cells are growing is assayed for production of monoclonal antibodies directed against the antigen. In certain embodiments, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a typical source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., 1990, Nature, 348:552-554. Clackson et al., 1991, Nature, 352:624-628, and Marks et al., 1991, J. Mol. Biol., 222:581-597 describe the isolation of murine and human antibodies, respectively, using phage libraries. Subsequent publications describe the production of high affinity (nM range) human antibodies by chain shuffling (Marks et al., 1992, Bio/Technology, 10:779-783), as well as combinatorial infection and in vivo recombination as a strategy for constructing very large phage libraries (Waterhouse et al., 1993, Nuc. Acids. Res., 21:2265-2266). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., 1984, Proc. Natl. Acad. Sci. USA, 81:6851), or by covalently joining to the immunoglobulin coding sequence all or part of the coding sequence for non-immunoglobulin material (e.g., protein domains).

Typically such non-immunoglobulin material is substituted for the constant domains of an antibody, or is substituted for the variable domains of one antigen-combining site of an antibody to create a chimeric bivalent antibody comprising one antigen-combining site having specificity for an antigen and another antigen-combining site having specificity for a different antigen.

c. Humanized and Human Antibodies

A humanized antibody has one or more amino acid residues from a source that is non-human. The non-human amino acid residues are often referred to as “import” residues, and are typically taken from an “import” variable domain. Humanization can be performed generally following the method of Winter and co-workers (Jones et al., 1986, Nature, 321:522-525; Riechmann et al., 1988, Nature, 332:323-327; Verhoeyen et al., 1988, Science, 239:1534-1536), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in non-human, for example, rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., 1987, J Immunol., 151:2296; Chothia et al., 1987, J. Mol. Biol., 196:901). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA, 89:4285; Presta et al., 1993, J. Immunol., 151:2623).

It is further important that antibodies be humanized with retention of high affinity for the antigen and other favorable biological properties. To achieve this goal, according to a one embodiment, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., 1993, Proc. Natl. Acad. Sci. USA, 90:2551; Jakobovits et al., 1993, Nature, 362:255-258; Bruggermann et al., 1993, Year in Immuno., 7:33; and Duchosal et al., 1992, Nature 355:258. Human antibodies can also be derived from phage-display libraries (Hoogenboom et al., 1991, J. Mol. Biol., 227:381; Marks et al., J. Mol. Biol., 1991, 222:581-597; Vaughan et al., 1996, Nature Biotech 14:309).

i. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

d. Multispecific Antibodies

Multispecific antibodies have binding specificities for at least two different antigens. While such molecules may only bind two antigens (e.g., bispecific antibodies, BsAbs), antibodies with additional specificities such as trispecific antibodies are encompassed by this expression when used herein. Examples of BsAbs include those with one antigen binding site directed against IL-1β and another antigen binding site directed against IL-18. In some embodiments, the BsAbs comprise a first binding specificity for IL-1β or IL-18 and a second binding specificity for an activating receptor having a cytoplasmic ITAM motif. An ITAM motif structure possesses two tyrosines separate by a 9-11 amino acid spacer. A general consensus sequence is YxxL/I(x)₆₋₈YxxL (Isakov, N., 1997, J. Leukoc. Biol., 61:6-16). Exemplary activating receptors include FcεRI, FcγRIII, FcγRI, FcγRIIA, and FcγRIIC. Other activating receptors include, e.g., CD3, CD2, CD10, CD161, DAP-12, KAR, KARAP, FcεRII, Trem-1, Trem-2, CD28, p44, p46, B cell receptor, LMP2A, STAM, STAM-2, GPVI, and CD40 (See, e.g., Azzoni, et al., 1998, J. Immunol. 161:3493; Kita, et al., 1999, J. Immunol. 162:6901; Merchant, et al., 2000, J. Biol. Chem. 74:9115; Pandey, et al., 2000, J. Biol. Chem. 275:38633; Zheng, et al., 2001, J. Biol. Chem. 276:12999; Propst, et al., 2000, J. Immunol. 165:2214).

In one embodiment, a multispecifc antibody comprises a first binding specificity for IL-1β and a second binding specificity for IL-18. Multispecific, including bispecific, antibodies can be prepared as full length antibodies or antibody fragments (for example, F(ab′)₂ bispecific antibodies). Bispecific antibodies may additionally be prepared as knobs-in-holes or hingeless antibodies. Bispecific antibodies are reviewed in Segal et al., 2001, J. Immunol. Methods 248:1-6.

Methods for making bispecific antibodies are known in the art. Traditional production of full length bispecific antibodies is based on the coexpression of two immunoglobulin heavy chain-light chain pairs, where the two chains have different specificities (Millstein et al., 1983, Nature, 305:537-539). Because of the random assortment of immunoglobulin heavy and light chains, these hybridomas (quadromas) produce a potential mixture of 10 different antibody molecules, of which only one has the correct bispecific structure. Purification of the correct molecule, usually done by affinity chromatography steps, is rather cumbersome, and the product yields are low. Similar procedures are disclosed in WO 93/08829, and in Traunecker et al., 1991, EMBO J., 10:3655-3659.

According to a different approach, antibody variable domains with the desired binding specificities (antibody-antigen combining sites) are fused to immunoglobulin constant domain sequences. The fusion can be with an immunoglobulin heavy chain constant domain, comprising at least part of the hinge, CH2, and CH3 regions. In certain embodiments, the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three antibody fragments in embodiments when unequal ratios of the three antibody chains used in the construction provide the optimum yields. It is, however, possible to insert the coding sequences for two or all three antibody chains in one expression vector when the expression of at least two antibody chains in equal ratios results in high yields or when the ratios are of no particular significance.

In another embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile method of separation. This approach is disclosed in WO 94/04690. For further details of methods for generating bispecific antibodies, see, for example, Suresh et al., 1986, Methods in Enzymology, 121:210.

According to another approach described in WO96/27011, the interface between a pair of antibody molecules can be engineered to maximize the percentage of heterodimers that are recovered from recombinant cell culture. In one embodiment, the interface comprises at least a part of the CH3 domain of an antibody constant domain. In this method, one or more small amino acid side chains from the interface of the first antibody molecule are replaced with larger side chains (for example, tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed, for example, in U.S. Pat. No. 4,676,980, along with a number of cross-linking techniques.

Antibodies with more than two valencies are also contemplated. For example, trispecific antibodies can be prepared According to Tutt et al., 1991, J. Immunol. 147: 60.

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to two targets, e.g., IL-1β as well as IL-18 (see, US 2008/0069820, for example).

e. Antibodies with Variant Hinge Regions

The antibodies as described herein may also comprise variant heavy chains, for example as described in application Ser. No. 10/697,995, filed Oct. 30, 2003. Antibodies comprising variant heavy chains comprise an alteration of at least one disulfide-forming cysteine residue, such that the cysteine residue is incapable of forming a disulfide linkage. In one aspect, said cysteine(s) is of the hinge region of the heavy chain (thus, such a hinge region is referred to herein as a “variant hinge region” and may additionally be referred to as “hingeless”).

In some aspects, such immunoglobulins lack the complete repertoire of heavy chain cysteine residues that are normally capable of forming disulfide linkages, either intermolecularly (such as between two heavy chains) or intramolecularly (such as between two cysteine residues in a single polypeptide chain). Generally, the disulfide linkage formed by the cysteine residue(s) that is altered (i.e., rendered incapable of forming disulfide linkages) is one that, when not present in an antibody, does not result in a substantial loss of the normal physicochemical and/or biological characteristics of the immunoglobulin. In certain embodiments, the cysteine residue that is rendered incapable of forming disulfide linkages is a cysteine of the hinge region of a heavy chain.

An antibody with variant heavy chains or variant hinge region is generally produced by expressing in a host cell an antibody in which at least one, at least two, at least three, at least four, or between two and eleven inter-heavy chain disulfide linkages are eliminated, and recovering said antibody from the host cell. Expression of said antibody can be from a polynucleotide encoding an antibody, said antibody comprising a variant heavy chain with reduced disulfide linkage capability, followed by recovering said antibody from the host cell comprising the polynucleotide. In one embodiment, the heavy chain comprises a variant hinge region of an immunoglobulin heavy chain, wherein at least one cysteine of said variant hinge region is rendered incapable of forming a disulfide linkage.

It is further anticipated that any cysteine in an immunoglobulin heavy chain can be rendered incapable of disulfide linkage formation, similarly to the hinge cysteines described herein, provided that such alteration does not substantially reduce the biological function of the immunoglobulin. For example, IgM and IgE lack a hinge region, but each contains an extra heavy chain domain; at least one (in some embodiments, all) of the cysteines of the heavy chain can be rendered incapable of disulfide linkage formation so long as it does not substantially reduce the biological function of the heavy chain and/or the antibody which comprises the heavy chain.

Heavy chain hinge cysteines are well known in the art, as described, for example, in Kabat, 1991, “Sequences of proteins of immunological interest,” supra. As is known in the art, the number of hinge cysteines varies depending on the class and subclass of immunoglobulin. See, for example, Janeway, 1999, Immunobiology, 4th Ed., (Garland Publishing, NY). For example, in human IgGIs, two hinge cysteines are separated by two prolines, and these are normally paired with their counterparts on an adjacent heavy chain in intermolecular disulfide linkages. Other examples include human IgG2 that contains 4 hinge cysteines, IgG3 that contains 11 hinge cysteines, and IgG4 that contains 2 hinge cysteines.

Accordingly, in certain embodiments, methods include expressing in a host cell an immunoglobulin heavy chain comprising a variant hinge region, where at least one cysteine of the variant hinge region is rendered incapable of forming a disulfide linkage, allowing the heavy chain to complex with a light chain to form a biologically active antibody, and recovering the antibody from the host cell.

Alternative embodiments include those where at least 2, 3, or 4 cysteines are rendered incapable of forming a disulfide linkage; where from about two to about eleven cysteines are rendered incapable; and where all the cysteines of the variant hinge region are rendered incapable.

Light chains and heavy chains constituting antibodies as produced according to methods described herein may be encoded by a single polynucleotide or by separate polynucleotides.

Cysteines normally involved in disulfide linkage formation can be rendered incapable of forming disulfide linkages by any of a variety of methods known in the art, or those that would be evident to one skilled in the art in view of the criteria described herein. For example, a hinge cysteine can be substituted with another amino acid, such as serine that is not capable of disulfide bonding. Amino acid substitution can be achieved by standard molecular biology techniques, such as site directed mutagenesis of the nucleic acid sequence encoding the hinge region that is to be modified. Suitable techniques include those described in Sambrook et al., 1989, Molecular Cloning: A Laboratory Manual, 2nd Ed., Other techniques for generating an immunoglobulin with a variant hinge region include synthesizing an oligonucleotide that encodes a hinge region, where the codon for the cysteine to be substituted is replaced with a codon for the substitute amino acid. This oligonucleotide can then be ligated into a vector backbone comprising other appropriate antibody sequences, such as variable regions and Fc sequences, as appropriate.

In another embodiment, a hinge cysteine can be deleted. Amino acid deletion can be achieved by standard molecular biology techniques, such as site directed mutagenesis of the nucleic acid sequence encoding the hinge region that is to be modified. Suitable techniques include those described in Sambrook et al., supra. Other techniques for generating an immunoglobulin with a variant hinge region include synthesizing an oligonucleotide comprising a sequence that encodes a hinge region in which the codon for the cysteine to be modified is deleted. This oligonucleotide can then be ligated into a vector backbone comprising other appropriate antibody sequences, such as variable regions and Fc sequences, as appropriate.

f. Bispecific Antibodies Formed Using “Protuberance-Into-Cavity” Strategy

In some embodiments, bispecific antibodies are formed using a “protuberance-into-cavity” strategy, also referred to as “knobs into holes” that serves to engineer an interface between a first and second polypeptide for hetero-oligomerization. In one embodiment, the interface comprises at least a part of the CH3 domain of an antibody constant domain. The “knobs into holes” mutations in the CH3 domain of an Fc sequence has been reported to greatly reduce the formation of homodimers (See, for example, Merchant et al., 1998, Nature Biotechnology, 16:677-681). “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). Where a suitably positioned and dimensioned protuberance or cavity exists at the interface of either the first or second polypeptide, it is only necessary to engineer a corresponding cavity or protuberance, respectively, at the adjacent interface. The protuberance and cavity can be made by synthetic means such as altering the nucleic acid encoding the polypeptides or by peptide synthesis. For further description of knobs into holes, see U.S. Pat. Nos. 5,731,168; 5,807,706; 5,821,333.

In some embodiments “knobs into holes” technology is used to promote heterodimerization to generate full-length bispecific anti-FcγRIIB and anti-“activating receptor” (e.g., IgER) antibody. In one embodiment, constructs were prepared for the anti-FcγIIB component (e.g., p5A6.11.Knob) by introducing the “knob” mutation (T366W) into the Fc region, and the anti-IgER component (e.g., p22E7.11.Hole) by introducing the “hole” mutations (T366S, L368A, Y407V). In another embodiment, constructs are prepared for the anti-FcγIIB component (e.g., p5A6.11.Hole) by introducing a “hole” mutation into its Fc region, and the anti-IgER component (e.g., p22E7.11.Knob) by introducing a “knob” mutation in its Fc region such as by the procedures disclosed herein or the procedures disclosed by Merchant et al., (1998), supra, or in U.S. Pat. Nos. 5,731,168; 5,807,706; 5,821,333.

A general method of preparing a heteromultimer using the “protuberance-into-cavity” strategy comprises expressing, in one or separate host cells, a polynucleotide encoding a first polypeptide that has been altered from an original polynucleotide to encode a protuberance, and a second polynucleotide encoding a second polypeptide that has been altered from the original polynucleotide to encode the cavity. The polypeptides are expressed, either in a common host cell with recovery of the heteromultimer from the host cell culture, or in separate host cells, with recovery and purification, followed by formation of the heteromultimer. In some embodiments, the heteromultimer formed is a multimeric antibody, for example a bispecific antibody. See also U.S. patent application Ser. No. 13/092,708 filed 22 Apr. 2011.

In some embodiments, antibodies combine a knobs into holes strategy with variant hinge region constructs to produce hingeless bispecific antibodies.

### Vectors, Host Cells and Recombinant Methods

Also provided are isolated polynucleotides encoding the antibodies as disclosed herein, vectors and host cells comprising the polynucleotides, and recombinant techniques for the production of the antibodies.

For recombinant production of the antibody, a polynucleotide encoding the antibody is isolated and inserted into a replicable vector for further cloning (amplification of the DNA) or for expression. DNA encoding the antibody is readily isolated and sequenced using conventional procedures, for example, by using oligonucleotide probes capable of binding specifically to genes encoding the antibody. Many vectors are available. The vector components generally include, but are not limited to, one or more of the following: a signal sequence, an origin of replication, one or more marker genes, an enhancer element, a promoter, and a transcription termination sequence.

(i) Signal Sequence Component

The antibodies described herein may be produced recombinantly, not only directly, but also as fusion antibodies with heterologous antibodies. In one embodiment, the heterologous antibody is a signal sequence or other antibody having a specific cleavage site at the N-terminus of the mature protein or antibody. The heterologous signal sequence selected typically is one that is recognized and processed (i.e., cleaved by a signal peptidase) by the host cell. For prokaryotic host cells that do not recognize and process the native antibody signal sequence, the signal sequence is substituted by a prokaryotic signal sequence selected, for example, from the group of the alkaline phosphatase, penicillinase, 1pp, or heat-stable enterotoxin II leaders. For yeast secretion the native signal sequence may be substituted by, e.g., the yeast invertase leader, α factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available. The DNA for such precursor region is ligated in reading frame to DNA encoding the antibody.

In another embodiment, production of antibodies can occur in the cytoplasm of the host cell, and therefore does not require the presence of secretion signal sequences within each cistron. In that regard, immunoglobulin light and heavy chains are expressed, folded, and assembled to form functional immunoglobulins within the cytoplasm. Certain host strains (for example, the E. coli trxB strains) provide cytoplasm conditions that are favorable for disulfide bond formation, thereby permitting proper folding and assembly of expressed protein subunits (Proba and Plukthun, 1995, Gene, 159:203).

g. (ii) Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 μplasmid origin is suitable for yeast, and various viral origins (SV40, polyoma, adenovirus, VSV, or BPV) are useful for cloning vectors in mammalian cells. Generally, the origin of replication component is not needed for mammalian expression vectors (the SV40 origin may typically be used only because it contains the early promoter).

h. (iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, for example, primate metallothionein genes, adenosine deaminase, ornithine decarboxylase, and the like.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp 1 gene present in the yeast plasmid YRp7 (Stinchcomb et al., 1979, Nature, 282:39). The trp1 gene provides a selection marker for a mutant strain of yeast lacking the ability to grow in tryptophan, for example, ATCC No. 44076 or PEP4-1. Jones, 1977, Genetics, 85:12. The presence of the trp1 lesion in the yeast host cell genome then provides an effective environment for detecting transformation by growth in the absence of tryptophan. Similarly, Leu2-deficient yeast strains (for example, strains having ATCC accession number 20,622 or 38,626) are complemented by known plasmids bearing the Leu2 gene.

In addition, vectors derived from the 1.6 μm circular plasmid pKDl can be used for transformation of Kluyveromyces yeasts. Alternatively, an expression system for large-scale production of recombinant calf chymosin was reported for K. lactis. See Van den Berg, 1990, Bio/Technology, 8:135. Stable multi-copy expression vectors for secretion of mature recombinant human serum albumin by industrial strains of Kluyveromyces have also been disclosed. See Fleer et al., 1991, Bio/Technology, 9:968-975.

i. (iv) Promoter Component

Expression and cloning vectors usually contain a promoter that is recognized by the host organism and is operably linked to the antibody nucleic acid. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phos-phate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., 1982, Nature 297:598-601 on expression of human β-interferon cDNA in mouse cells under the control of a thymidine kinase promoter from herpes simplex virus. Alternatively, the rous sarcoma virus long terminal repeat can be used as the promoter.

j. (v) Enhancer Element Component

Transcription of a DNA encoding an antibody by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297:17-18 on enhancing elements for activation of eukaryotic promoters. The enhancer may be spliced into the vector at a position 5′ or 3′ to the antibody-encoding sequence, but is typically located at a site 5′ from the promoter.

k. (vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO94/11026 and the expression vector disclosed therein.

l. (vii) Modulation of Translational Strength

Immunoglobulins described herein can also be expressed from an expression system in which the quantitative ratio of expressed light and heavy chains can be modulated in order to maximize the yield of secreted and properly assembled full length antibodies. Such modulation is accomplished by simultaneously modulating translational strengths for light and heavy chains.

One technique for modulating translational strength is disclosed in Simmons et al., U.S. Pat. No. 5,840,523 and Simmons et al., 2002, J. Immunol. Methods, 263: 133-147. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are typical. Alterations in the TIR can include, for example, alterations in the number or spacing of Shine-Dalgarno sequences, along with alterations in the signal sequence. One exemplary method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al, 1992, METHODS: A Companion to Methods in Enzymol., 4:151-158.

In some embodiments, a set of vectors is generated with a range of TIR strengths for each cistron therein. This limited set provides a comparison of expression levels of each chain as well as the yield of full length products under various TIR strength combinations. TIR strengths can be determined by quantifying the expression level of a reporter gene as described in detail in Simmons et al., U.S. Pat. No. 5,840,523 and Simmons et al., 2002, J. Immunol. Methods, 263: 133-147. In certain embodiments, the translational strength combination for a particular pair of TIRs within a vector is represented by (N-light, M-heavy), wherein N is the relative TIR strength of light chain and M is the relative TIR strength of heavy chain. For example, (3-light, 7-heavy) means the vector provides a relative TIR strength of about 3 for light chain expression and a relative TIR strength of about 7 for heavy chain expression. Based on the translational strength comparison, the desired individual TIRs are selected to be combined in expression vector constructs.

m. (viii) Selection and Transformation of Host Cells

Suitable host cells for cloning or expressing the DNA in the vectors herein are the prokaryote, yeast, or higher eukaryote cells described above. Suitable prokaryotes for this purpose include Archaebacteria and Eubacteria, such as Gram-negative or Gram-positive organisms, for example, Enterobacteriaceae such as Escherichia, e.g., E. coli, Enterobacter, Envinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710, published 12 Apr. 1989), Pseudomonas such as P. aeruginosa, and Streptomyces. One E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. It is also useful for the host cell to secrete minimal amounts of proteolytic enzymes, and additional protease inhibitors may desirably be incorporated in the cell culture. Prokaryotic host cells may also comprise mutation(s) in the thioredoxin and/or glutathione pathways.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein, particularly for transfection of Spodoptera frugiperda cells. Plant cell cultures of cotton, corn, potato, soybean, petunia, tomato, and tobacco can also be utilized as hosts.

Vertebrate host cells are widely used, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., 1980, Proc. Natl. Acad. Sci. USA 77:4216); mouse sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; mouse myeloma cells, such as NSO (e.g. RCB0213, 1992, Bio/Technology 10:169) and SP2/0 cells (e.g. SP2/0-Ag14 cells, ATCC CRL 1581); rat myeloma cells, such as YB2/0 cells (e.g. YB2/3HL.P2.G11.16Ag.20 cells, ATCC CRL 1662); and a human hepatoma line (Hep G2). CHO cells are an exemplary cell line for practicing the methods described herein, with CHO-K1, DUK-B11, CHO-DP12, CHO-DG44 (Somatic Cell and Molecular Genetics 12:555 (1986)), and Lec13 being exemplary host cell lines. In the case of CHO-K1, DUK-B11, DG44 or CHO-DP12 host cells, these may be altered such that they are deficient in their ability to fucosylate proteins expressed therein.

Hybridoma cells may also be used. The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line that produces antibodies such as, for example, quadromas (See, for example, Milstein et al., 1983, Nature, 537:3053). The hybrid cell lines can be of any species, including human and mouse.

In some embodiments, the mammalian cell is a non-hybridoma mammalian cell, which has been transformed with exogenous isolated nucleic acid encoding the antibody of interest. By “exogenous nucleic acid” or “heterologous nucleic acid” is meant a nucleic acid sequence that is foreign to the cell, or homologous to the cell but in a position within the host cell nucleic acid in which the nucleic acid is ordinarily not found.

n. (ix) Culturing the Host Cells

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10 (Sigma), Minimal Essential Medium ((MEM), (Sigma), RPMI-1640 (Sigma)), and Dulbecco's Modified Eagle's Medium ((DMEM), Sigma) are suitable for culturing the host cells. In addition, any of the media described in Ham et al., 1979, Meth. Enz. 58:44, Barnes et al., 1980, Anal. Biochem. 102:255, U.S. Pat. No. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

All culture medium typically provides at least one component from one or more of the following categories:
1) an energy source, usually in the form of a carbohydrate such as glucose;
2) all essential amino acids, and usually the basic set of twenty amino acids plus cystine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

In certain embodiments, the culture medium is free of serum, e.g. less than about 5%, or less than 1%, or 0 to 0.1% serum, and other animal-derived proteins. However, they can be used if desired. In one embodiment, the cell culture medium comprises excess amino acids. The amino acids that are provided in excess may, for example, be selected from Asn, Asp, Gly, Ile, Leu, Lys, Met, Ser, Thr, Trp, Tyr, and Val. In one embodiment, Asn, Asp, Lys, Met, Ser, and Trp are provided in excess. For example, amino acids, vitamins, trace elements and other media components at one or two times the ranges specified in European Patent EP 307,247 or U.S. Pat. No. 6,180,401 may be used. These two documents are incorporated by reference herein.

For the culture of the mammalian cells expressing the desired protein and capable of adding the desired carbohydrates at specific positions, numerous culture conditions can be used paying particular attention to the host cell being cultured. Suitable culture conditions for mammalian cells are well known in the art (W. Louis Cleveland et al., 1983, J. Immunol. Methods 56:221-234) or can be easily determined by the skilled artisan (see, for example, Animal Cell Culture: A Practical Approach 2^{nd} Ed., Rickwood, D. and Hames, B. D., eds. Oxford University Press, New York (1992)), and vary according to the particular host cell selected.

o. (x) Antibody Purification

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., 1992, Bio/Technology 10: 163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being an exemplary purification technique. The suitability of protein A as an affinity ligand depends on the species and isotype of any immunoglobulin Fc region that is present in the antibody. Protein A can be used to purify antibodies that are based on human γ1, γ2, or γ4 heavy chains (Lindmark et al., 1983, J. Immunol. Meth. 62:1-13). Protein G is recommended for all mouse isotypes and for human γ3 (Guss et al., 1986, EMBO J. 5:15671575). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

In one embodiment, the glycoprotein may be purified using adsorption onto a lectin substrate (e.g. a lectin affinity column) to remove fucose-containing glycoprotein from the preparation and thereby enrich for fucose-free glycoprotein.

p. (xi) Antibody Activity Assays

The immunoglobulins can be characterized for their physical/chemical properties and biological functions by various assays known in the art. In one aspect, it is important to compare the selectivity of an antibody to bind the immunogen versus other binding targets.

In certain embodiments, the immunoglobulins produced herein are analyzed for their biological activity. In some embodiments, the immunoglobulins are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays. Illustrative antigen binding assays are provided below in the Examples section.

The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography, and papain digestion. Methods for protein quantification are well known in the art. For example, samples of the expressed proteins can be compared for their quantitative intensities on a Coomassie-stained SDS-PAGE. Alternatively, the specific band(s) of interest (e.g., the full length band) can be detected by, for example, western blot gel analysis and/or AMES-RP assay.

### Pharmaceutical Formulations

Therapeutic formulations of the antibody/antibodies can be prepared by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) antibody; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active compound as necessary for the particular indication being treated, for example, those with complementary activities that do not adversely affect each other. For instance, the formulation may further comprise another antibody or a chemotherapeutic agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### Non-Therapeutic Uses for Antibodies

Antibodies may be used as an affinity purification agent. In this process, the antibody is immobilized on a solid phase such a Sephadex™ resin or filter paper, using methods well known in the art. The immobilized antibody is contacted with a sample containing the antigen to be purified, and thereafter the support is washed with a suitable solvent that will remove substantially all the material in the sample except the antigen to be purified, which is bound to the immobilized antibody. Finally, the support is washed with another suitable solvent, such as glycine buffer, pH 5.0, that will release the antigen from the antibody.

The antibody may also be useful in diagnostic assays, e.g., for detecting expression of an antigen of interest in specific cells, tissues, or serum. For diagnostic applications, the antibody typically will be labeled with a detectable moiety. Numerous labels are available which can be generally grouped into the following categories:
(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.
(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.
(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone and H. Van Vunakis), Academic press, New York, 73:147-166 (1981).

Examples of enzyme-substrate combinations include, for example:
1) Horseradish peroxidase (HRPO) utilizes hydrogen peroxide to oxidize a dye precursor (e.g., orthophenylene diamine (OPD) or 3,3′,5,5′-tetramethyl benzidine hydrochloride (TMB));
2) alkaline phosphatase (AP) with para-Nitrophenyl phosphate as chromogenic substrate; and
3) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment, the antibody need not be labeled, and the presence thereof can be detected using a labeled antibody which binds to the antibody.

The antibody may be employed in any known assay method, such as competitive binding assays, direct and indirect sandwich assays, and immunoprecipitation assays. Zola, Monoclonal Antibodies: A Manual of Techniques, pp. 47-158 (CRC Press, Inc. 1987).

The antibody may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P or ³⁵S) so that the antigen or cells expressing it can be localized using immunoscintiography.

### In Vivo (Therapeutic) Uses for Antibodies

Therapeutic antibodies have been developed and approved for treatment of a variety of diseases. In certain embodiments, anti-IL-1β and/or anti-IL-18 antibody/antibodies are co-administered with a therapeutic agent to enhance the function of the therapeutic agent.

For the prevention or treatment of disease, the appropriate dosage of antibody will depend on the type of disease to be treated, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 15 mg/kg (e.g., 0.1-20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The antibody composition should be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat a disease or disorder. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as used hereinbefore or about from 1 to 99% of the heretofore employed dosages.

For example, for treating autoimmune diseases where there is the involvement of an inflammatory cell (e.g., leukocyte) adhesion, migration and activation, such as rheumatoid arthritis and lupus, the antibody herein can be co-administered with, e.g., anti-LFA-1 antibody (such as an anti-CD11a or anti-CD18 antibody) or an anti-ICAM antibody such as ICAM-1, -2, or -3. Additional agents for treating rheumatoid arthritis in combination with the antibody herein include Enbrel™, DMARDS, e.g., methotrexate, and NSAIDs (non-steroidal anti-inflammatory drugs). More than one of such other active agents than the antibody herein may also be employed. Additionally, insulin can be used for treating diabetes, anti-IgE for asthma, anti-CD11a for psoriasis, anti-alpha4beta7 and growth hormone (GH) for inflammatory bowel disease.

### Articles of Manufacture

In another embodiment, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody. The label or package insert indicates that the composition is used for treating the condition of choice. Moreover, the article of manufacture may comprise (a) a first container with a composition contained therein, wherein the composition comprises an antibody; and (b) a second container with a composition contained therein, wherein the composition comprises a further cytotoxic or otherwise therapeutic agent. The article of manufacture in this embodiment may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Therapeutic antibody compositions generally are placed into a container having a sterile access port, for example, an intravenous solution bag or vial having a stopper pierceable by a hypodermic injection needle.

Also provided is an article of manufacture and kit containing materials useful for the treatment of inflammatory bowel disease, for example, CD or UC. The article of manufacture comprises a container with a label. Suitable containers include, for example, bottles, vials, and test tubes. The containers may be formed from a variety of materials such as glass or plastic. The container holds a composition comprising the antibody described herein. The active agent in the composition is the particular antibody. The label on the container indicates that the composition is used for the treatment or prevention of a particular disease or disorder, and may also indicate directions for in vivo, such as those described above.

In certain embodiments, the kit comprises the container described above and a second container comprising a buffer. It may further include other materials desirable from a commercial and user standpoint, including other buffers, diluents, filters, needles, syringes, and package inserts with instructions for use.

### General Biomarker Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Primers, oligonucleotides and polynucleotides employed in the present invention can be generated using standard techniques known in the art.

Gene expression biomarkers associated with predicting responsiveness of IBD patients including patient suffering from UC or Crohn's Disease to certain therapeutic agents are provided herein. These expression levels of the mRNA or individual proteins encoded by the genes constitute biomarkers for predicting responsiveness to IBD therapeutic agents, UC therapeutic agents, and/or Crohn's Disease therapeutic agents. Accordingly, the invention disclosed herein is useful in a variety of settings, e.g., in methods and compositions related to diagnosis and therapy of inflammatory bowel diseases.

### Detection of Gene Expression Levels

Nucleic acid, according to any of the methods described herein may be RNA transcribed from genomic DNA or cDNA generated from RNA or mRNA. Nucleic acid may be derived from a vertebrate, e.g., a mammal. A nucleic acid is said to be “derived from” a particular source if it is obtained directly from that source or if it is a copy of a nucleic acid found in that source.

Nucleic acid includes copies of the nucleic acid, e.g., copies that result from amplification. Amplification may be desirable in certain instances, e.g., in order to obtain a desired amount of material for detecting variations. The amplicons may then be subjected to a variation detection method, such as those described below, to determine expression of certain genes.

Levels of mRNA may be measured and quantified by various methods well-known to those skilled in the art, including use of commercially available kits and reagents. One such method is polymerase chain reaction (PCR). Another method, for quantitative use, is real-time quantitative PCR, or qPCR. See, e.g., “PCR Protocols, A Guide to Methods and Applications,” (M. A. Innis et al., eds., Academic Press, Inc., 1990); “Current Protocols in Molecular Biology” (F. M. Ausubel et al., eds., 1987, and periodic updates); and “PCR: The Polymerase Chain Reaction”, (Mullis et al., eds., 1994).

A microarray is a multiplex technology that typically uses an arrayed series of thousands of nucleic acid probes to hybridize with, e.g, a cDNA or cRNA sample under high-stringency conditions. Probe-target hybridization is typically detected and quantified by detection of fluorophore-, silver-, or chemiluminescence-labeled targets to determine relative abundance of nucleic acid sequences in the target. In typical microarrays, the probes are attached to a solid surface by a covalent bond to a chemical matrix (via epoxy-silane, amino-silane, lysine, polyacrylamide or others). The solid surface is for example, glass, a silicon chip, or microscopic beads. Various microarrays are commercially available, including those manufactured, for example, by Affymetrix, Inc. and Illumina, Inc.

A biological sample may be obtained using certain methods known to those skilled in the art. Biological samples may be obtained from vertebrate animals, and in particular, mammals. In certain instances, a biological sample is synovial tissue, serum or peripheral blood mononuclear cells (PBMC). By screening such body samples, a simple early diagnosis can be achieved for diseases such as ulcerative colitis and Crohn's Disease. In addition, the progress of therapy can be monitored more easily by testing such body samples for variations in expression levels of target nucleic acids (or encoded polypeptides).

Subsequent to the determination that a subject, or the tissue or cell sample comprises a gene expression signature or relative levels of certain biomarkers disclosed herein, it is contemplated that an effective amount of an appropriate therapeutic agent may be administered to the subject to treat the particular disease in the subject, e.g., UC or Crohn's Disease. Clinical diagnosis in mammals of the various pathological conditions described herein can be made by the skilled practitioner. Clinical diagnostic techniques are available in the art which allow, e.g., for the diagnosis or detection of inflammatory bowel diseases in a mammal, e.g., ulcerative colitis and Crohn's Disease.

### Diagnostic Assay Kits

For use in the applications described or suggested herein, diagnostic assay kits or articles of manufacture are also provided. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a polynucleotide comprising one or more genes of a gene expression signature. Where the kit utilizes nucleic acid hybridization to detect the target nucleic acid, the kit may also have containers containing nucleotide(s) for amplification of the target nucleic acid sequence and/or a container comprising a reporter means, such as a biotin-binding protein, such as avidin or streptavidin, bound to a reporter molecule, such as an enzymatic, florescent, or radioisotope label.

Diagnostic assay kits will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above. Other optional components in the kit include one or more buffers (e.g., block buffer, wash buffer, substrate buffer, and the like), other reagents such as substrate (e.g., chromogen) which is chemically altered by an enzymatic label, epitope retrieval solution, control samples (positive and/or negative controls), control slide(s) etc.

### Methods of Marketing

The invention herein also encompasses a method for marketing a therapeutic agent or a pharmaceutically acceptable composition thereof comprising promoting to, instructing, and/or specifying to a target audience, the use of the agent or pharmaceutical composition thereof for treating a patient or patient population with a particular disease, e.g., UC or Crohn's Disease, from which a sample has been obtained showing a gene expression signature or levels of serum biomarkers or peripheral blood biomarkers or detection of tissue biomarkers as disclosed herein.

Marketing is generally paid communication through a non-personal medium in which the sponsor is identified and the message is controlled. Marketing for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The marketing of the diagnostic method herein may be accomplished by any means. Examples of marketing media used to deliver these messages include television, radio, movies, magazines, newspapers, the internet, and billboards, including commercials, which are messages appearing in the broadcast media.

The type of marketing used will depend on many factors, for example, on the nature of the target audience to be reached, e.g., hospitals, insurance companies, clinics, doctors, nurses, and patients, as well as cost considerations and the relevant jurisdictional laws and regulations governing marketing of medicaments and diagnostics. The marketing may be individualized or customized based on user characterizations defined by service interaction and/or other data such as user demographics and geographical location.

The foregoing written specification and following examples are considered to be sufficient to enable one skilled in the art to practice the invention. Various modifications of the invention in addition to those shown and described herein will become apparent to those skilled in the art from the foregoing description and following examples and fall within the scope of the appended claims.

It is understood that the application of the teachings of the present invention to a specific problem or situation will be within the capabilities of one having ordinary skill in the art in light of the teachings contained herein.

Further details of the invention are illustrated by the following non-limiting Examples. The disclosures of all citations in the specification are expressly incorporated herein by reference.

### EXAMPLES

Commercially available reagents referred to in the examples were used according to manufacturer's instructions unless otherwise indicated.

### Materials and Methods

Human Resection Tissue Samples

Patients undergoing bowel resection for ulcerative colitis, Crohn's disease, diverticulitis or colon cancer gave informed consent to participate in the study. Prior to surgery, blood samples were obtained and either shipped fresh to Genentech overnight or processed according to standard clinical laboratory procedures and stored as serum or plasma. Following surgery, tissue in excess of that required for clinical analysis was harvested. A small portion was snap frozen for protein analysis and the remaining tissue was shipped to Genentech overnight.

Primary intestinal subepithelial myofibroblasts (MFs) were isolated from patient bowel resection samples. In brief, surface epithelial cells were detached from mucosal samples by three sequential treatments with 1 mmol EDTA, and the tissue samples, denuded of epithelial cells, were cultured (at 37° C. and 5% CO2) in 10% fetal calf serum (FCS)-RPMI (Gibco-Invitrogen, Calsbad, US). MFs migrated out of the subepithelial regions of the lamina propria to establish colonies in tissue culture dishes. After removal of the tissue samples, primary MFs proliferated to establish monolayers that could be maintained over several passages. The cultures were confirmed as primary MFs by flow cytometry (alpha-smooth muscle actin-positive, vimentin-positive, desmin-negative). Primary MFs were cultured in DMEM supplemented with high glucose with 10% FBS, 1% nonessential amino acids and an antibiotic cocktail (100 ug/ml Penicillin/Strep, 20 ug/ml Gentamicin, 2.5 ug/ml Amphotercin B and 1 ug/ml Metronidazole) and used for experiments at passages 3 to 5.

MFs (from three different patients) were plated at 65,000 cells per well (6 well plate) overnight and were stimulated with supplemented DMEM as noted above with or without added IL-1β (10 ng/ml) (Cat#201-LB-CF, R&D systems, Minneapolis, Minn.) or TNFα (10 ng/ml) (Cat#210-TA-CF, R&D systems, Minneapolis, Minn.) for 6 h or 24 h at 37°, depending on the experiment, then collected for RNA isolation. RNA was isolated using RNeasy columns (Qiagen), according to the manufacturer's protocol, including an on-column DNAse digestion step and then prepared for microarray (Agilent, Santa Clara, Calif.).

Snap frozen resected tissue samples were processed in a liquid nitrogen pre-cooled Bessman tissue pulverizer (Spectrum Laboratories, Rancho Dominguez, Calif.; catalog #189475). Tissue pieces were homogenized with 1 ml lysis buffer (1×TBS, 1% Triton-X 100, 2× complete protease inhibitor cocktail) in FastPrep®-24 Instrument with 2×45 s shaking. Homogenized tissues were then centrifuged for 10 minutes at 14,000 g in a cold microfuge. Supernatants were aliquoted and stored at −80° C.

Microarray Analysis

RNA was amplified and labeled using the Quick Amp labeling kit (Agilent, Santa Clara, Calif.) to generate labeled cRNA from 1 ug of total RNA. Experimental samples were labeled with Cy5; Universal Human Reference RNA (Stratagene, La Jolla, Calif.) was used for the reference channel and was labeled with Cy3. Cy5 and Cy3 labeled cRNA was competitively hybridized to the two-color Whole Human Genome 4×44K gene expression microarray platform. Hybridized microarrays were washed according to the manufacturer's protocol (Agilent, Santa Clara, Calif.) and all feature intensities were collected using the Agilent Microarray Scanner. TIFF images of scanned slides were analyzed using Feature Extraction Software (Agilent, Santa. Clara, Calif.), protocol GE2-v5_95 (Agilent, Santa Clara, Calif.). All data were reported as log₂ values of the dye-normalized Cy5/Cy3 ratios.

Identification of IL1β and TNFα Induced Genes

A linear model was fit to each of the IL-1β, TNFα, and media control treatments and time points. We identified genes that showed both a change from baseline and a difference between treatments of at least 1.5 fold (false discovery rate, or FDR=0.001). Genes uniquely upregulated by IL-1β and TNF-α were evaluated for overlap.

Identification of Fibrosis-Related Genes

RNA was isolated from full thickness punch biopsies taken from resected tissue samples and subjected to microarray analysis. Fibrosis associated genes were identified by comparing gene expression between biopsies from patients undergoing resection for fibrostenosis vs. all other patients. Because the fibrosis co-occurs with inflammation in Crohn's disease, many of the fibrostenotic biopsies showed varying levels of immune infiltration. To estimate the amount of immune cell infiltration, we calculated the first eigenvector of the gene expression values for a panel of cell type-specific gene sets (see Abbas et al., Genes Immun. 6(4):319-31(2005)). This eigenvector was used as an estimate of the degree of infiltration for that cell type within that sample. These estimates were used as covariates in a linear model that also accounted for diagnosis, fibrosis, and biopsy location. Genes were selected based on this model at an FDR of 0.1.

qRT-PCR

RNA was isolated from in vitro MF stimulation experiments, biopsies from tissue resection samples, and from OCT sections adjacent to those used for histological analysis for use in the qPCR studies. First strand cDNA was generated from 200 ng of total RNA using iScript (Bio-Rad, Hercules, Calif.). 12.5 ng of cDNA was pre-amplified using the Taqman PreAmp Master Mix (Life Technologies, Carlsbad, Calif.) along with diluted Taqman assays (Life Technologies, Carlsbad, Calif.), according to the manufacturer's suggestion. qPCR was performed using the Biomark HD system (Fluidigm 96.96 format) and a panel of 48 Taqman assays comprised of genes identified from the microarray data as well as several house-keeping genes. Resultant data was normalized to GAPDH to yield ΔCₜ values.

ELISA

Supernatants from the stimulation were collected and assayed for GCSF (Cat# HSTCS0, R&D systems, Minneapolis, Minn.) and Amphiregulin (ab99975, Abcam, Cambridge, Mass.) by ELISA following the manufacturer's protocol and guidelines.

IL-11 (ab100551, Abcam, Cambridge, Mass.), IL1Ra (DRA00B, R&D systems, Minneapolis, Minn.), IL18BP (DY119, R&D systems, Minneapolis, Minn.), Amphiregulin (ab99975, Abcam, Cambridge, Mass.) and GCSF (HSTCS0, R&D systems, Minneapolis, Minn.) were measured in patient serum samples following the manufacturer's protocol and guidelines.

IL-1β (HSLB00C, R&D systems, Minneapolis, Minn.), IL1Ra (DRA00B, R&D systems, Minneapolis, Minn.), IL18 (7620, R&D systems, Minneapolis, Minn.) and IL18BP (DY119, R&D systems, Minneapolis, Minn.), were measured in tissue lysates following the manufacturer's protocol and guidelines.

### Example 1

### IL-1β-Induced Expression of Fibrosis-Associated Genes in Human Primary Intestinal Myofibroblasts

Activation of intestinal subepithelial myofibroblasts (MFs) may play a key role in intestinal fibrosis through increased deposition of collagen and extracellular matrix proteins. Because the role of inflammatory cytokines, such as IL-1β, in this process is not well understood, we investigated the expression of genes in tissue samples from patients undergoing bowel resection and studied IL-1β gene induction in primary MFs. The characteristics of the patients studied are shown in Table 1.

TABLE 1
Patient Characteristics

| | | Past surgery | Biologic therapy |
|---|---|---|---|
| | Reason for surgery | (Y/N) | (Y/N) |
| Non-IBD | Diverticulitis (n = 4) | 1/3 | 0/4 |
| (n = 10) | Colon cancer (n = 6) | 2/4 | 0/6 |
| CD | Non-obstructive | 4/1 | 2/3 |
| (n = 15) | inflammatory (n = 5) | | |
| | Fibrostenosis (n = 10) | 5/5 | 7/3 |
| UC | Dysplasia (n = 2) | 0/2 | 0/2 |
| (n = 18) | Refractory disease (n = 16) | 0/16 | 13/3 |

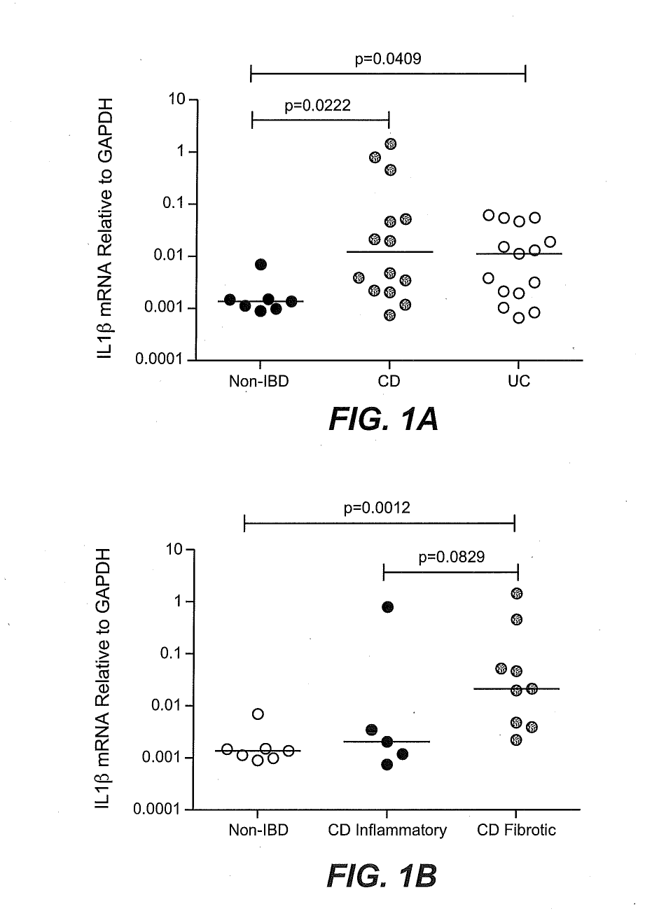

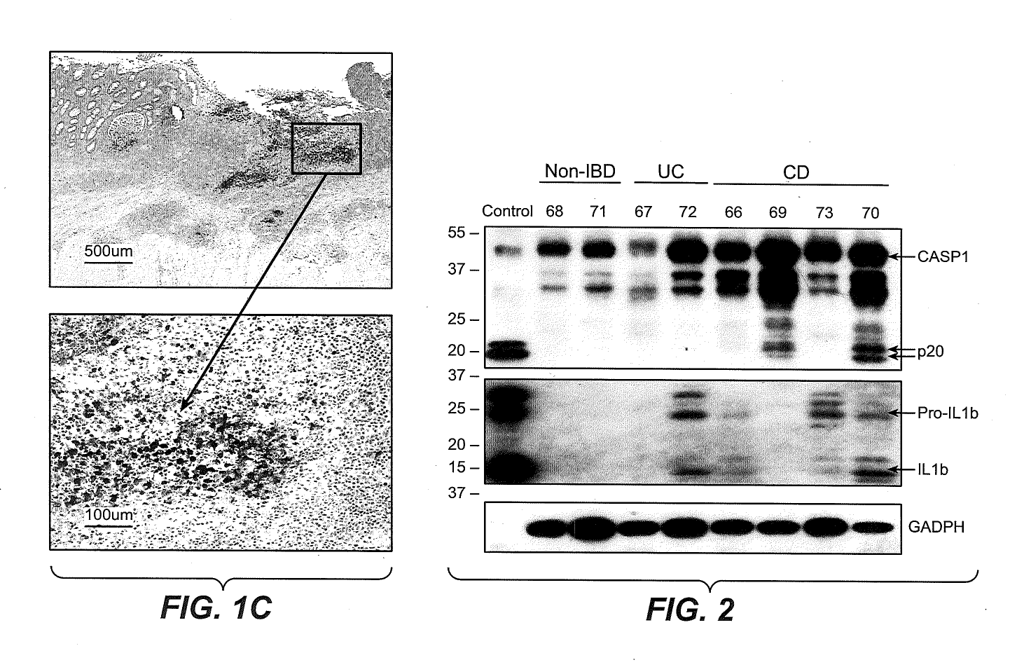

We first examined whether there were differences in IL-1β mRNA levels in tissue from IBD and non-IBD patients undergoing bowel resection. As shown in FIG. 1A, the level of IL-1β mRNA in tissue samples from CD patients and from UC patients was significantly higher than the level of IL-1β mRNA in samples from non-IBD patients (median relative expression level in CD patients and in UC patients was approximately 0.01 compared to approximately 0.001 in non-IBD patients, p=0.0222 and p=0.0409, respectively). Furthermore, the level of IL-1β mRNA in CD patients undergoing surgery for fibrostenotic disease trended higher than in CD patients undergoing surgery for inflammatory disease. We found the difference in IL-1β mRNA levels compared to non-IBD patient tissue to be even greater as shown in FIG. 1B (median relative expression level in fibrotic tissue from CD patients was approximately 0.015 compared to approximately 0.001 in tissue from non-IBD CD patients, p=0.0012). In addition, we observed increased IL-1β protein expression by immunohistochemistry in resected CD tissue (FIG. 1C).

We also performed immunoblot analysis of resected tissue obtained from non-IBD patients, UC patients, or CD patients. As shown in FIG. 2, this analysis revealed that proteins associated with inflammasome activation, CASP1 and p20, along with pro-IL1β and mature IL-1β were highly expressed in tissues from CD patients, in particular, and in UC pateints, but barely detectable in non-IBD tissues. These data provided evidence for increased expression of IL-1β in fibrotic tissue in CD patients compared to either non-fibrotic or inflammatory tissue from CD patients or to non-IBD colonic tissue. Flow cytometry and qRT-PCR analysis demonstrated that IL1R1 and TNFR1 were present on primary intestinal sub-epithelial MFs isolated from resected tissues (data not shown).

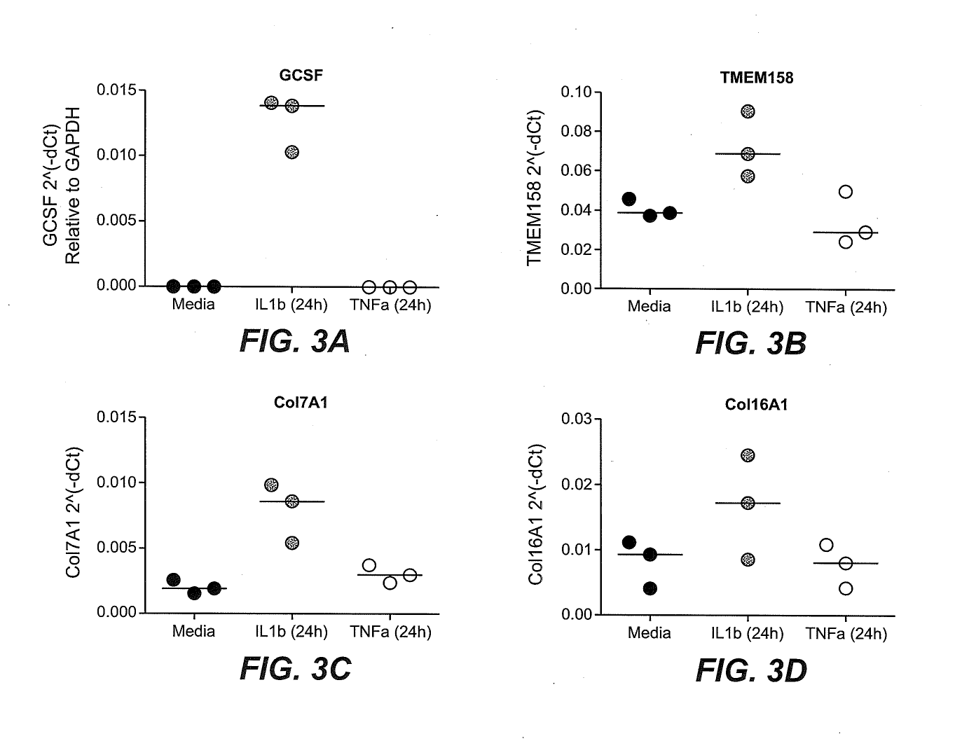

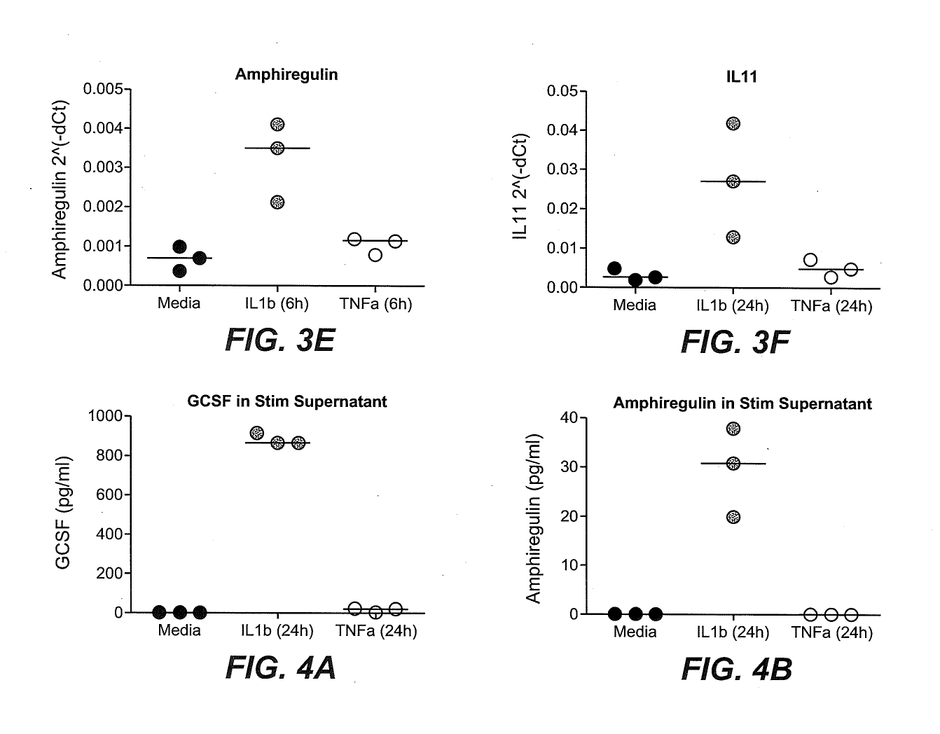

Next, we investigated genes regulated in primary MFs by IL-1β but not by TNFα using the methods described above. We found a group of genes including collagen that are specifically induced by IL-1β but not by TNFα in MF. The genes showing the most induction by IL-1β treatment and no induction by TNFα treatment as assessed by microarray analysis are shown in Table 2 below. Gene expression was verified by qRT-PCR of stimulated MFs as shown in FIG. 3. IL-1β treatment, but not TNFα treatment, stimulated expression of GCSF (FIG. 3A), TMEM158 (FIG. 3B), Col7A1 (FIG. 3C), Col16A1 (FIG. 3D), amphiregulin (FIG. 3E), and IL-11 (FIG. 3F), thereby verifying the microarray findings.

TABLE 2
Microarray analysis of MFs.

| Symbol | Induced by IL1b | Induced by TNFa |
|---|---|---|
| CSF3 | 12.38 | No |
| IL24 | 4.50 | No |
| SERPINB3 | 3.43 | No |
| IL11 | 3.03 | No |
| SERPINB4 | 2.75 | No |
| AMIGO2 | 2.58 | No |
| SERPINB7 | 2.46 | No |
| ABAT | 2.46 | No |
| PF4 | 2.19 | No |
| STEAP2 | 2.03 | No |
| ELN | 2.00 | No |
| CCL4 | 1.99 | No |
| VEGFA | 1.96 | No |
| TMEM158 | 1.95 | No |
| DACT1 | 1.92 | No |
| KCNMB4 | 1.88 | No |
| COL16A1 | 1.85 | No |
| PDLIM4 | 1.85 | No |
| TGFBR1 | 1.62 | No |
| KCNE1L | 1.57 | No |
| HIF1A | 1.56 | No |
| SLC25A45 | 1.55 | No |
| OSMR | 1.51 | No |
| P4HA2 | 1.51 | No |
| ELF3 | 1.48 | No |
| TGIF1 | 1.32 | No |
| AREG | 1.16 | No |

Supernatants from MF cultures stimulated with media, IL-1β or TNFα were analyzed by ELISA for GCSF and amphiregulin. As shown in FIGS. 4A-B, GCSF and amphiregulin proteins were increased in supernatants after IL-1β treatment, but not TNFα or media treatment, consistent with the qRT-PCR results.

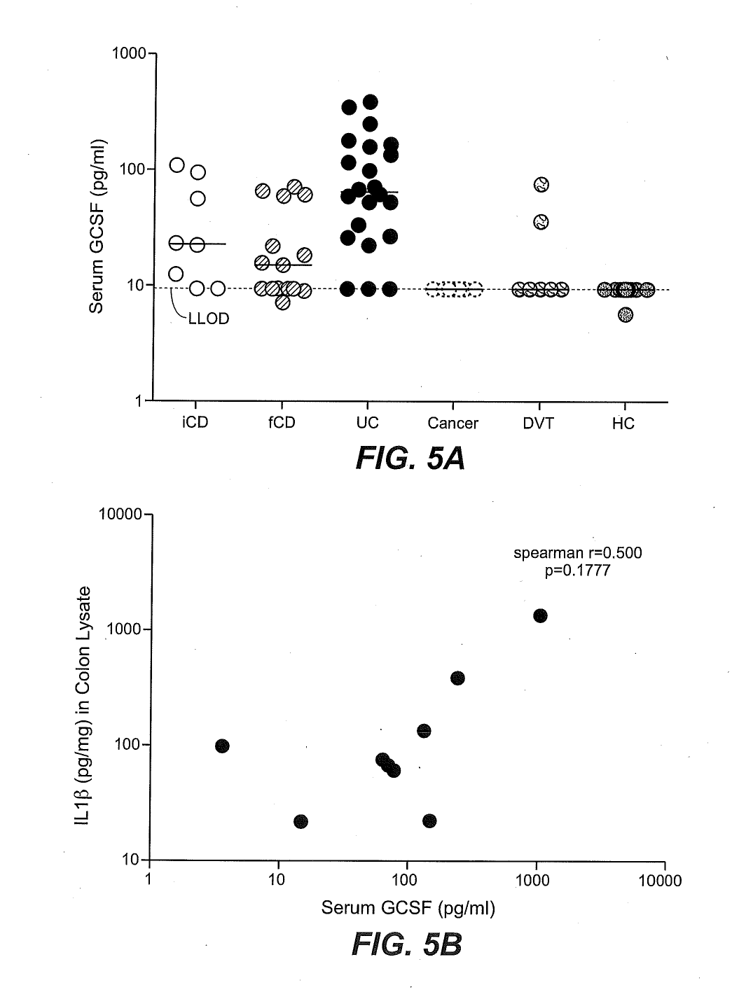

Because the above results demonstrated that we could measure and quantify GCSF and amphiregulin levels in cell supernatants by ELISA, and that we could observe a similar increase in protein levels following IL-1β stimulation as observed by measuring RNA expression, we tested the feasibility of measuring GCSF in human serum. FIG. 5A shows that serum GCSF levels were upregulated in both CD and UC patients compared to healthy controls. While we were unable to detect serum GCSF in any cancer or healthy control patient, we could readily detect serum GCSF in a number of fCD patients and iCD patients. Only two DVT patients demonstrated detectable levels of GCSF in their serum. Using the same samples, we correlated the level of IL-1β in intestinal tissue lysates with serum GCSF and found that patients with higher IL-1β expression in intestinal tissue lysates had higher detectable levels of serum GCSF (FIG. 5B, spearman r=0.500, p=0.1777). These data suggest that serum GCSF is a useful surrogate biomarker of IL-1β protein levels in intestinal tissues.

We found that multiple stromal and muscle genes were upregulated in fibrotic tissue compared to non-fibrotic tissue, for example, INHBA and LMCD1. In addition, extracellular matrix turnover and tissue remodeling genes (e.g., MMP3, PXDN), collagen genes (e.g., Col7A1, Col5A2, Col12A1, Col18A1), and Wnt signaling target genes (e.g., TMEM158, CHN1) were upregulated in fibrotic tissue compared to non-fibrotic tissue. Table 3 below shows the identification of fibrosis genes determined by microarray analysis; the protein location is also indicated in the table. We further found IL-1β-regulated genes from several pathways including cytokines (e.g., IL24, IL11), collagens (e.g., Col7A1, Col16A1), and WNT signaling genes (e.g., TMEM158, DACT1) in MFs. TNF-α induced genes in MFs included chemokine (e.g., IL-8, CXCL3) and adhesion molecules genes (e.g., ICAM1). IL-1β stimulation of MFs was observed to induce collagen and WNT signaling genes that overlap with genes upregulated in intestinal fibrosis (e.g., TMEM158, Col7A1). In contrast, no genes associated with intestinal fibrosis were found to be uniquely upregulated by TNF-α in MFs.

TABLE 3
Fibrosis genes

| | Fold | | |
|---|---|---|---|
| Symbol | Change | Function | Protein location |
| MMP3 | 7.41 | Matrix associated | Secreted |
| | | protein | |
| INHBA | 3.25 | Stromal cells | Secreted |
| COL5A2 | 2.73 | Collagen | Secreted |
| CHN1 | 2.50 | WNT signaling | Cytosol |
| LMCD1 | 2.48 | Muscle cells | Unknown |
| COL12A1 | 2.45 | Collagen | Secreted |
| COL7A1 | 2.43 | Collagen | Secreted |
| COL18A1 | 2.38 | Collagen | Secreted |
| TMEM158 | 2.38 | WNT signaling | Membrane protein |
| FAM65C | 2.30 | Stromal cells | Unknown |
| IGFBP5 | 2.28 | Growth | Secreted |
| THY1 | 2.28 | Myofibroblast | Membrane protein |
| TMEM132A | 2.28 | WNT signaling | Membrane protein |
| PXDN | 2.13 | Matrix-associated | Secreted |
| | | protein | |
| GPR68 | 2.08 | Muscle cells | Membrane protein |
| TWIST1 | 2.08 | EMT | Nuclear, transcription |
| | | | factor |
| COL4A1 | 2.07 | Collagen | Secreted |
| SERPINH1 | 2.03 | WNT signaling | Secreted |
| AEBP1 | 2.00 | Muscle cells | Secreted, transcription |
| | | | factor |
| NAB2 | 1.99 | Growth | Nuclear, transcription |
| | | | factor |
| TMEM45A | 1.81 | WNT signaling | Membrane protein |
| TMEM121 | 1.72 | WNT signaling | Membrane protein |
| VIM | 1.68 | filament | Cytosol |
| NOTCH4 | 1.68 | growth | Membrane protein |
| TIMP2 | 1.67 | Inhibitor of | Secreted |
| | | metalloproteinase | |

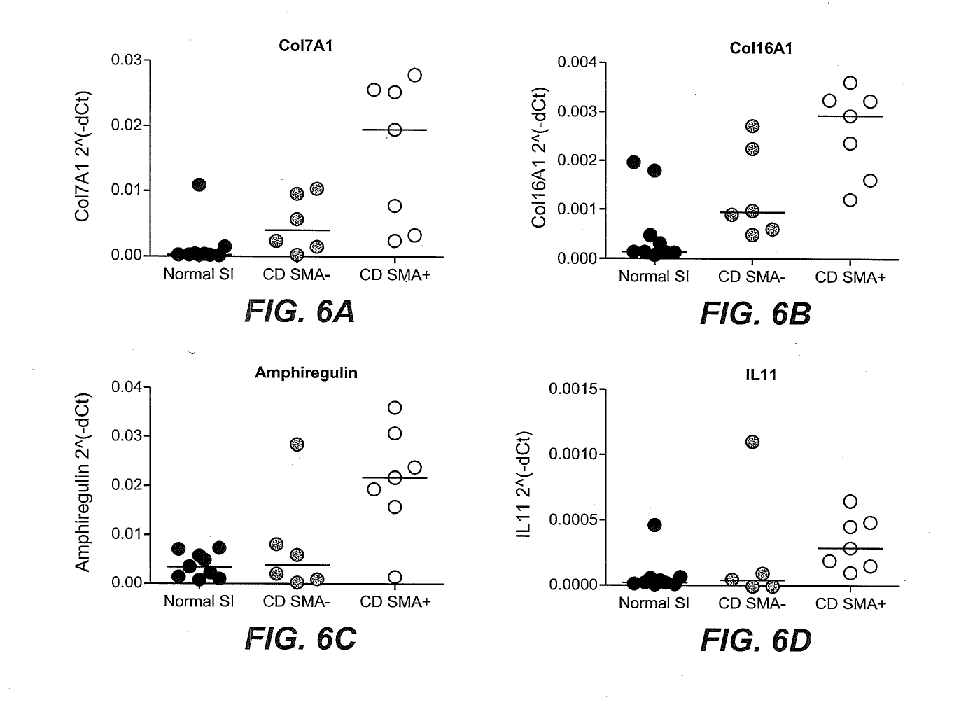

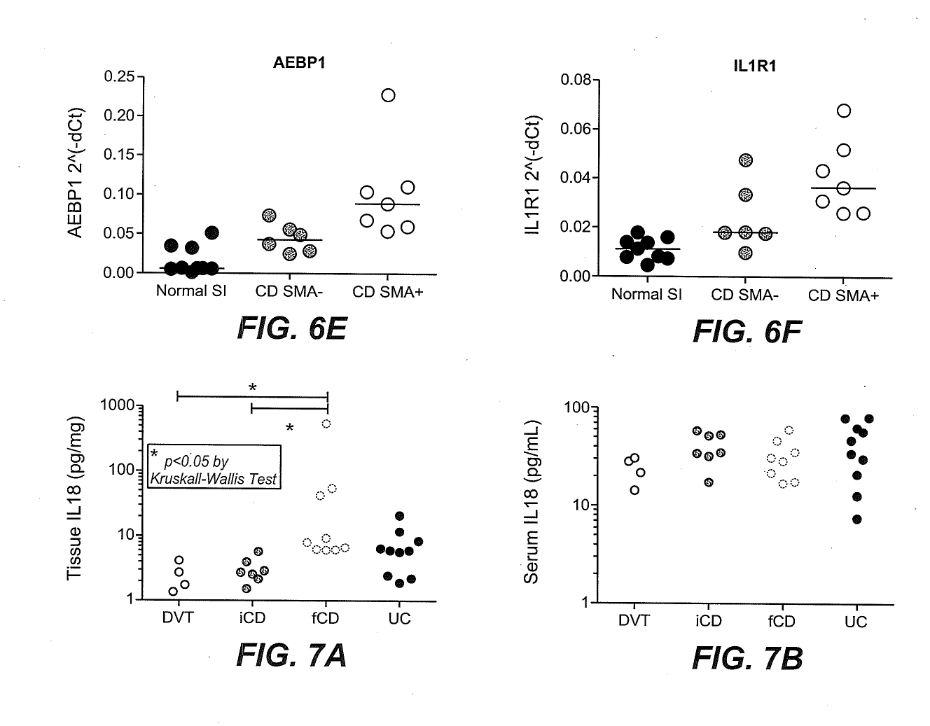

To confirm our microarray finding, we analyzed OCT sections adjacent to those used for SMA staining by IHC. Sections were scored by a pathologist for SMA; typically, SMA+ samples are considered to have evidence of fibrosis. The qRT-PCR results are shown in FIGS. 6A-F. We found that collagen subtypes 7A1 and 16A1, along with amphiregulin, IL-11, AEBP1 and IL1R1 had increased expression in CD SMA+ sections in comparison to CD SMA− sections or control sections, demonstrating that these IL-1β induced genes are upregulated in fibrotic tissues.

### Example 2

### Additional Biomarkers

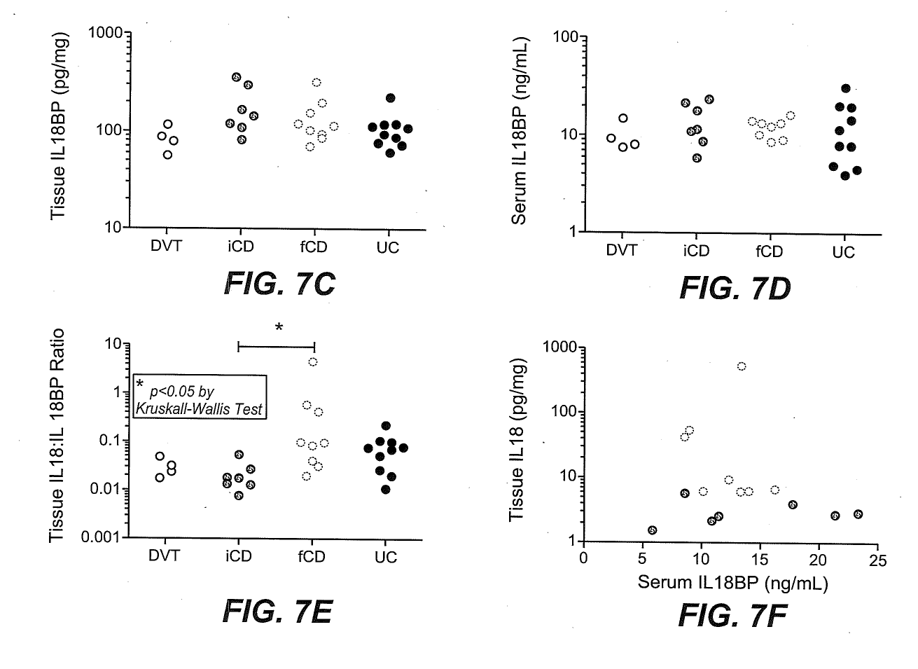

Using the patient cohort described in Table 4, we investigated additional biomarkers that could be useful to differentiate fibrotic/fibrostenotic (fCD) and inflammatory CD (iCD), using UC and DVT as comparators. Using matched intestinal tissue lysates and serum samples, we assayed each sample for IL18 and IL18BP levels by ELISA. We found that fCD patients expressed somewhat higher levels of IL18 in intestinal tissue compared to iCD patients (FIG. 7A, p<0.05 by Kruskall-Wallis test) but there was no significant difference in serum levels (FIG. 7B). We found no significant differences in the levels of IL18BP between tissue and serum in any of the patient groups (FIGS. 7C and 7D). We also examined the ratio of IL18 to IL18BP in intestinal tissue lysate. As shown in FIG. 7E, the IL18:IL18BP ratio was somewhat higher in fCD patients compared to iCD patients (p<0.05 by Kruskall-Wallis test). In addition, FIG. 7F shows the intestinal IL18 levels plotted against serum IL18BP for the fCD patients and the iCD patients.

TABLE 4
Patient Characteristics

| | | Past surgery | Biologic therapy |
|---|---|---|---|
| | Reason for surgery | (Y/N) | (Y/N) |
| Non-IBD | Diverticulitis (n = 4) | 1/3 | 0/4 |
| (n = 6) | Colon cancer (n = 2) | 1/1 | 0/2 |
| CD | Non-obstructive | 5/2 | 5/2 |
| (n = 15) | inflammatory (n = 7) | | |
| | Fibrostenosis (n = 9) | 4/5 | 8/1 |
| UC | Refractory disease (n = 10) | 5/5 | 8/2 |

### REFERENCES

Arend, W. P., G. Palmer, and C. Gabay. 2008. IL-1, IL-18, and IL-33 families of cytokines. Immunol Rev. 223:20-38.
Baldassano, R. N., J. P. Bradfield, D. S. Monos, C. E. Kim, J. T. Glessner, T Casalunovo, E. C. Frackelton, F. G. Otieno, S. Kanterakis, J. L. Shaner, R. M. Smith, A. W. Eckert, L. J. Robinson, C. C. Onyiah, D. J. Abrams, R. M. Chiavacci, R. Skraban, M. Devoto, S. F. Grant, and H. Hakonarson. 2007. Association of the T300A non-synonymous variant of the ATG16L1 gene with susceptibility to paediatric Crohn's disease. Gut. 56:1171-3.
Cadwell, K., J. Y. Liu, S. L. Brown, H. Miyoshi, J. Loh, J. K. Lennerz, C. Kishi, W. Kc, J. A. Carrero, S. Hunt, C. D. Stone, E. M. Brunt, R. J. Xavier, B. P. Sleckman, E. Li, N. Mizushima, T. S. Stappenbeck, and H. W.t. Virgin. 2008. A key role for autophagy and the autophagy gene Atg16l1 in mouse and human intestinal Paneth cells. Nature. 456:259-63.
Carter, K. W., J. Hung, B. L. Powell, S. Wiltshire, B. T. Foo, Y. C. Leow, B. M. McQuillan, M. Jennens, P. A. McCaskie, P. L. Thompson, J. P. Beilby, and L. J. Palmer. 2008. Association of Interleukin-1 gene polymorphisms with central obesity and metabolic syndrome in a coronary heart disease population. Hum Genet. 124:199-206.
Cassel, S. L., S. Joly, and F. S. Sutterwala. 2009. The NLRP3 inflammasome: A sensor of immune danger signals. Semin Immunol.
Ferrero-Miliani, L., O. H. Nielsen, P. S. Andersen, and S. E. Girardin. 2007. Chronic inflammation: importance of NOD2 and NALP3 in interleukin-1 beta generation. Clin Exp Immunol. 147:227-35.
Kowluru, R. A., and S. Odenbach. 2004. Role of interleukin-1 beta in the pathogenesis of diabetic retinopathy. Br J Ophthalmol. 88:1343-7.
Kuballa, P., A. Huett, J. D. Rioux, M. J. Daly, and R. J. Xavier. 2008. Impaired autophagy of an intracellular pathogen induced by a Crohn's disease associated ATG16L1 variant. PLoS One. 3:e3391.
Larsen, C. M., M. Faulenbach, A. Vaag, A. Volund, J. A. Ehses, B. Seifert, T. Mandrup-Poulsen, and M. Y. Donath. 2007. Interleukin-1-receptor antagonist in type 2 diabetes mellitus. N Engl J Med. 356:1517-26.
Lewis, E. C., and C. A. Dinarello. 2006. Responses of IL-18- and IL-18 receptor-deficient pancreatic islets with convergence of positive and negative signals for the IL-18 receptor. Proc Natl Acad Sci USA. 103:16852-7.
Ludwiczek, O., A. Kaser, D. Novick, C. A. Dinarello, M. Rubinstein, and H. Tilg. 2005. Elevated systemic levels of free interleukin-18 (IL-18) in patients with Crohn's disease. Eur Cytokine Netw. 16:27-33.
Ludwiczek, O., E. Vannier, I. Borggraefe, A. Kaser, B. Siegmund, C. A. Dinarello, and H. Tilg. 2004. Imbalance between interleukin-1 agonists and antagonists: relationship to severity of inflammatory bowel disease. Clin Exp Immunol. 138:323-9.
Monteleone, G., F. Trapasso, T. Parrello, L. Biancone, A. Stella, R. Iuliano, F. Luzza, A. Fusco, and F. Pallone. 1999. Bioactive IL-18 expression is up-regulated in Crohn's disease. J Immunol. 163:143-7.
Perrier, S., F. Darakhshan, and E. Hajduch. 2006. IL-1 receptor antagonist in metabolic diseases: Dr Jekyll or Mr Hyde? FEBS Lett. 580:6289-94.
Saitoh, T., N. Fujita, M. H. Jang, S. Uematsu, B. G. Yang, T. Satoh, H. Omori, T. Noda, N. Yamamoto, M. Komatsu, K. Tanaka, T. Kawai, T. Tsujimura, O. Takeuchi, T. Yoshimori, and S. Akira. 2008. Loss of the autophagy protein Atg16L1 enhances endotoxin-induced IL-1 beta production. Nature. 456:264-8.
Sandberg, J. O., A. Andersson, D. L. Eizirik, and S. Sandler. 1994. Interleukin-1 receptor antagonist prevents low dose streptozotocin induced diabetes in mice. Biochem Biophys Res Commun. 202:543-8.
Sidhu, S. S., B. Li, Y. Chen, F. A. Fellouse, C. Eigenbrot, and G. Fuh. 2004. Phage-displayed antibody libraries of synthetic heavy chain complementarity determining regions. J Mol Biol. 338:299-310.
Ten Hove, T., A. Corbaz, H. Amitai, S. Aloni, I. Belzer, P. Graber, P. Drillenburg, S. J. van Deventer, Y. Chvatchko, and A. A. Te Velde. 2001. Blockade of endogenous IL-18 ameliorates TNBS-induced colitis by decreasing local TNF-alpha production in mice. Gastroenterology. 121:1372-9.
Villani, A. C., M. Lemire, G. Fortin, E. Louis, M. S. Silverberg, C. Collette, N. Baba, C. Libioulle, J. Belaiche, A. Bitton, D. Gaudet, A. Cohen, D. Langelier, P. R. Fortin, J. E. Wither, M. Sarfati, P. Rutgeerts, J. D. Rioux, S. Vermeire, T. J. Hudson, and D. Franchimont. 2009. Common variants in the NLRP3 region contribute to Crohn's disease susceptibility. Nat Genet. 41:71-6.

1. A method of diagnosing, or aiding in diagnosing, an inflammatory bowel disease in a subject, the method comprising:
(a) measuring in a biological sample obtained from the subject the expression level of one or a combination of genes, or the expression of one or a combination of proteins encoded by the one or the combinations of genes, selected from CSF3 (GCSF), IL-24, SERPINB3, SERPINB4, AMIGO2, SERPINB7, ABAT, PF4, STEAP2, ELN, CCL4, VEGFA, DACT1, KCNMB4, PDLIM4, TGFBR1, KCNE1L, HIF1A, SLC25A45, OSMR, P4HA2, ELF3, TGIF1, TMEM158, COL7A1, COL16A1, amphiregulin (AREG), and IL-11;
(b) comparing the expression level of the one or the combination of genes, or the one or the combination of proteins, measured in (a) to a reference level; and
(c) providing a diagnosis of inflammatory bowel disease when the level of the one or the combination of genes, or the one or the combination of proteins, measured in (a) is above the reference level.

2. The method of claim 1, wherein the one or the combination of genes, or the one or the combination of proteins encoded by the one or the combination of genes, is selected from CSF3 (GCSF), TMEM158, Col7A1, Col16A1, amphiregulin (AREG), IL-11.

3. The method of claim 2, wherein the one or the combination of genes, or the one or the combination of proteins encoded by the one or the combination of genes, is selected from CSF3 (GCSF) and amphiregulin.

4. A method of diagnosing, or aiding in diagnosing, an inflammatory bowel disease in a subject, the method comprising:
(a) measuring in a biological sample obtained from the subject the expression level of one or a combination of genes, or the expression of one or a combination of proteins encoded by the one or the combinations of genes, selected from IL-1β, CASP1, and p20;
(b) comparing the expression level of the one or the combination of genes, or the one or the combination of proteins, measured in (a) to a reference level; and
(c) providing a diagnosis of inflammatory bowel disease when the level of the one or the combination of genes, or the one or the combination of proteins, measured in (a) is above the reference level.

5. A method of diagnosing, or aiding in diagnosing, fibrotic Crohn's disease in a subject, the method comprising:
(a) measuring in a biological sample obtained from the subject the expression level of one or a combination of genes, or the expression of one or a combination of proteins encoded by the one or the combinations of genes, selected from COL7A1, COL16A1, amphiregulin, IL-11, AEBP1, and IL1R1;
(b) comparing the expression level of the one or the combination of genes, or the one or the combination of proteins, measured in (a) to a reference level; and
(c) providing a diagnosis of fibrotic Crohn's disease when the level of the one or the combination of genes, or the one or the combination of proteins, measured in (a) is above the reference level.

6. A method of diagnosing, or aiding diagnosing, fibrotic Crohn's disease in a subject, the method comprising:
(a) measuring in a biological sample obtained from the subject the expression level of one or a combination of genes, or the expression of one or a combination of proteins encoded by the one or the combinations of genes, selected from MMP3, INHBA, COL5A2, CHN1, LMCD1, COL12A1, COL7A1, COL18A1, TMEM158, FAM65C, IGFBP5, THY1, TMEM132A, PXDN, GPR68, TWIST1, COL4A1, SERPINH1, AEBP1, NAB2, TMEM45A, TMEM121, VIM, NOTCH4, and TIMP2;
(b) comparing the expression level of the one or the combination of genes, or the one or the combination of proteins, measured in (a) to a reference level; and
(c) providing a diagnosis of fibrotic Crohn's disease when the level of the one or the combination of genes, or the one or the combination of proteins, measured in (a) is above the reference level.

7. The method of claim 1 or claim 4, wherein the inflammatory bowel disease is ulcerative colitis or Crohn's disease.

8. The method of claim 5 or claim 6, wherein the fibrotic Crohn's disease is fibrostenotic Crohn's disease.

9. The method of any one of claims 1, 4, 5, and 6, wherein the biological sample is intestinal tissue.

10. The method of any one of claims 1, 4, 5, and 6, wherein the expression of the one or the combination of genes is measured using a PCR method or a microarray chip.

11. The method of any one of claims 1, 4, 5, and 6, wherein the expression of the one or the combination of proteins is measured using an immunoassay or an immunohistochemical assay.

12. The method of claim 11, wherein the immunoassay is an ELISA assay.

13. The method of any one of claims 1, 4, 5, and 6, wherein the reference level is obtained by measuring the expression level of the same one or combination of genes, or the same one or combination of proteins, in a biological sample obtained from a subject who does not have an inflammatory bowel disorder.

14. The method of claim 5 or claim 6, wherein the reference level is obtained by measuring the expression level of the same one or combination of genes, or the same one or combination of proteins, in nonfibrotic tissue.

15. A method of diagnosing, or aiding in diagnosing, a subtype of Crohn's disease in a subject, the method comprising:
(a) measuring in a biological sample obtained from intestinal tissue of the subject the expression level of IL18;
(b) comparing the expression level of IL18 measured in (a) to a reference level; and
(c) providing a diagnosis of fibrotic Crohn's disease when the level of IL18 measured in (a) is above the reference level.

16. The method of claim 15, wherein the reference level is obtained by measuring the expression level of IL18 in a biological sample obtained from intestinal tissue of a subject who does not have an inflammatory bowel disorder, or a subject who has inflammatory Crohn's disease, or a subject who has ulcerative colitis.

17. The method of claim 15, wherein the fibrotic Crohn's disease is fibrostenotic Crohn's disease.

18. The method of claim 15, wherein the expression level is measured using an immunoassay.

19. The method of claim 18, wherein the immunoassay is an ELISA assay.

20. A method of diagnosing, or aiding in diagnosing, an inflammatory bowel disease in a subject, the method comprising:
(a) measuring in a serum sample obtained from the subject the expression level of GCSF;
(b) comparing the expression level of GCSF measured in (a) to a reference level; and
(c) providing a diagnosis of inflammatory bowel disease when the level of GCSF measured in (a) is above the reference level.

21. The method of claim 20, wherein the inflammatory bowel disease is ulcerative colitis or inflammatory Crohn's disease, or fibrotic Crohn's disease.

22. The method of claim 21, wherein the fibrotic Crohn's disease is fibrostenotic Crohn's disease.

23. The method of claim 20, wherein the expression level of GCSF is measured using an immunoassay.

24. The method of claim 23, wherein the immunoassay is an ELISA assay.

25. The method of claim 20, wherein the reference level is obtained by measuring the expression level of GCSF in a serum sample obtained from a subject who does not have an inflammatory bowel disorder.

26. A method of treating inflammatory bowel disease in a patient comprising:
(a) measuring in a biological sample obtained from the patient the expression level of one or a combination of genes, or the expression of one or a combination of proteins encoded by the one or the combinations of genes, selected from CSF3 (GCSF), IL-24, SERPINB3, SERPINB4, AMIGO2, SERPINB7, ABAT, PF4, STEAP2, ELN, CCL4, VEGFA, DACT1, KCNMB4, PDLIM4, TGFBR1, KCNE1L, HIF1A, SLC25A45, OSMR, P4HA2, ELF3, TGIF1, TMEM158, COL7A1, COL16A1, amphiregulin (AREG), and IL-11;
(b) comparing the expression level of the one or the combination of genes, or the one or the combination of proteins, measured in (a) to a reference level; and
(c) administering an anti-IL-1β antibody, an anti-IL-18 antibody, or a multispecific anti-IL-1β/anti-IL-18 antibody to the patient when the level of the one or the combination of genes, or the one or the combination of proteins, measured in (a) is above the reference level.

27. The method of claim 26, wherein the one or the combination of genes, or the one or the combination of proteins encoded by the one or the combination of genes, is selected from CSF3 (GCSF), TMEM158, Col7A1, Col16A1, amphiregulin (AREG), IL-11.

28. The method of claim 27, wherein the one or the combination of genes, or the one or the combination of proteins encoded by the one or the combination of genes, is selected from CSF3 (GCSF) and amphiregulin.

29. A method of treating inflammatory bowel disease in a patient comprising:
(a) measuring in a biological sample obtained from the patient the expression level of one or a combination of genes, or the expression of one or a combination of proteins encoded by the one or the combinations of genes, selected from IL-1β, CASP1, and p20;
(b) comparing the expression level of the one or the combination of genes, or the one or the combination of proteins, measured in (a) to a reference level; and
(c) administering an anti-IL-1β antibody, an anti-IL-18 antibody, or a multispecific anti-IL-1β/anti-IL-18 antibody to the patient when the level of the one or the combination of genes, or the one or the combination of proteins, measured in (a) is above the reference level.

30. A method of treating inflammatory bowel disease in a patient comprising:
(a) measuring in a biological sample obtained from the subject the expression level of one or a combination of genes, or the expression of one or a combination of proteins encoded by the one or the combinations of genes, selected from MMP3, INHBA, COL5A2, CHN1, LMCD1, COL12A1, COL7A1, COL18A1, TMEM158, FAM65C, IGFBP5, THY1, TMEM132A, PXDN, GPR68, TWIST1, COL4A1, SERPINH1, AEBP1, NAB2, TMEM45A, TMEM121, VIM, NOTCH4, AEBP1, IL1R1, and TIMP2;
(b) comparing the expression level of the one or the combination of genes, or the one or the combination of proteins, measured in (a) to a reference level; and
(c) administering an anti-IL-1β antibody, an anti-IL-18 antibody, or a multispecific anti-IL-1β/anti-IL-18 antibody to the patient when the level of the one or the combination of genes, or the one or the combination of proteins, measured in (a) is above the reference level.

31. A method of treating inflammatory bowel disease in a patient comprising:
(a) measuring in a biological sample obtained from intestinal tissue of the patient the expression level of IL18;
(b) comparing the expression level of IL18 measured in (a) to a reference level; and
(c) administering an anti-IL-1β antibody, an anti-IL-18 antibody, or a multispecific anti-IL-1β/anti-IL-18 antibody to the patient when the level of the one or the combination of genes, or the one or the combination of proteins, measured in (a) is above the reference level.

32. A method of treating inflammatory bowel disease in a patient comprising:
(a) measuring in a serum sample obtained from the subject the expression level of GCSF;
(b) comparing the expression level of GCSF measured in (a) to a reference level; and
(c) administering an anti-IL-1β antibody, an anti-IL-18 antibody, or a multispecific anti-IL-1β/anti-IL-18 antibody to the patient when the level of GCSF measured in (a) is above the reference level.

33. The method of any one of claims 26, 29, 30, 31, and 32, wherein the inflammatory bowel disease is ulcerative colitis or inflammatory Crohn's disease, or fibrotic Crohn's disease.

34. The method of claim 33, wherein the fibrotic Crohn's disease is fibrostenotic Crohn's disease.

35. The method of any one of claims 26, 29, 30, 31, and 32, wherein the expression level is measured using a PCR method or a microarray chip.

36. The method of any one of claims 26, 29, 30, 31, and 32, wherein the expression level is measured using an immunoassay.

37. The method of claim 36, wherein the immunoassay is an ELISA assay.
